(19) United States
(12) Patent Application Publication
(10) Pub. No.: US 2015/0320833 A1
(43) Pub. Date: Nov. 12, 2015

(54) OSSIFICATION-INDUCING COMPOSITIONS AND METHODS OF USE THEREOF

(71) Applicants: Steven L. STICE, Athens, GA (US); John F. PERONI, Watkinsville, GA (US); Jennifer MUMAW, Watkinsville, GA (US); UNIVERSITY OF GEORGIA RESEARCH FOUNDATION, INC., Athens, GA (US)

(72) Inventors: Steven L. STICE, Athens, GA (US); John F. PERONI, Watkinsville, GA (US); Jennifer MUMAW, Watkinsville, GA (US)

(21) Appl. No.: 14/651,895

(22) PCT Filed: Dec. 13, 2013
(86) PCT No.: PCT/US13/75039

§ 371 Date: Jun. 12, 2015

Related U.S. Application Data
(60) Provisional application No. 61/736,764, filed on Dec. 13, 2012.

(51) Int. Cl. A61K 38/18; A61K 45/06; A61K 9/16; A61K 35/28

(52) U.S. Cl. CPC A61K 38/1875; A61K 35/28; A61K 45/06; A61K 9/1641; A61K 48/00

(57) ABSTRACT

Mesenchymal stem cells (MSCs) and uses thereof are provided. MSCs for inducing ossification and enhancing bone and/qr cartilage repair in a patient in need thereof are also provided. The method and compositions combine MSCs, at least one bone regeneration protein, such as bone morphogenetic protein (e.g. BMP-2), optionally in combination with additional cell growth factors including the components of a cell growth medium, further in combination with a biomaterial for delivery of the cells to the repair site in the patient are also provided.

### RELATED APPLICATIONS

This application claims the benefit of priority from provisional application serial no. U.S. 61/736,764, filed Dec. 13, 2012 of identical title, which application is incorporated by reference in its entirety herein.

### GRANT SUPPORT

This invention was made with government support under DARPA grant no. W911NF-09-1-0040 awarded by the U.S. Department of Defense. The government has certain rights in the invention.

### FIELD OF THE INVENTION

The present invention relates to the use of mesenchymal stem cells (MSCs). The MSCs can be used for inducing ossification and enhancing bone and/or cartilage repair in a subject. The method and compositions combine MSCs, at least one bone regeneration protein, such as, but not limited to, a bone morphogenetic protein (BMP), optionally in combination with additional cell growth factors including the components of a cell growth medium, further in combination with a biomaterial to the repair a site in the subject. It has been determined that the present compositions and methods can be used to induce ossification, repair bone defects, and/or cartilage defects with reduced side effects, such as, but not limited to, eliminating, reducing, preventing, or inhibiting, graft rejection and/or unfavorable immune reactions.

### BACKGROUND

Bone is the second most transplanted tissue behind blood transfusions. Autologous bone grafting is currently considered the gold standard for treating nonunions, but multiple features make it less than ideal for long bone nonunion treatment. The most promising graft donor site, the iliac crest, is available in limited quantity. As long bone nonunions can require up to 30 mls of marrow, the amount harvested from the iliac crest can be insufficient. Bone grafting presents considerable risks to patients by increased surgical times and blood loss, with ⅓ of patients experiencing chronic pain 24 months post-transplant and recipients being at increased risk for donor site instability and fractures. Additionally, large bone defects like those received by soldiers injured in combat often do not heal without surgical intervention ending often in an undesirable outcome, amputation.

Multiple members of the bone morphogenetic protein (BMP) family, a part of the TGF-β superfamily, have been shown to induce ossification (Yamaguchi 1996), and while they are named for their ability to form bone (Urist 1965), BMPs have additional roles in development (Yamamoto 2004). BMPs are involved in the establishment of dorsal ventral patterning and left right symmetry (Kishigami 2005), in cell fate determination though specification of ectoderm (Chang 1998), specification of limb bud identity (Sun 2000), in modulating proliferation (Wen 2004), and in apoptosis (Kawamura 2002). These versatile BMP signaling factors are secreted in an immature form and require cleavage to yield the active protein (Nohe 2004). This cleavage allows for the BMPs to bind as heterodimers and homodimers through di-sulfide linkage (Chen 2004), and for bone formation the heterodimer BMP2/BMP7 is an effective combination in inducing ossification (Koh 2008).

The binding of BMPs to their receptors is one of the mechanisms for such different functions throughout development (Kishigami 2005). BMPs bind to serine/threonine kinase type I and type II BMP receptors when in their active form. Without being bound to any specific theory, after binding their receptors SMAD 1/5/8-co-SMAD 4 signaling cascades are activated and translocated to the nucleus (Abe 2006). JNK, p38 (Guicheux 2003), and pI3 kinase (Osyczka 2005) are also involved with osteogenic signaling. In osteoblasts, reduction of either of these pathways reduces osteogenic marker expression and it is believed that cooperation between these pathways are involved in bone formation (Abe 2006).

BMPs have been shown to induce ossification (Yamaguchi 1996) and aid in bone matrix maturation and mineralization (van der Horst 2002) making them a potential substitute for bone grafts. Of the BMPs, BMP-2 and BMP-7 have been used in humans to promote spinal fusion, fracture healing and oral defects with differing reports of success (Mussano 2007). One drawback to BMPs is their rapid clearance if injected in solution (Lutolf 2003) and animal studies have shown that sustained exposure is required for efficient bone generation (Jeon 2008). Finding methods to produce constant amounts of BMPs at in situ sites of are of importance. Transducing cells with BMPs for injection have shown increased rate of ossification (Krebsbach 2000; Lee 2001; Dragoo 2003) (Bikram 2007; Olabisi 2010; Olabisi 2011).

Mesenchymal stem cells (MSCs) have been used in a wide variety of clinical and pre-clinical applications. Injections of mesenchymal stem cells into a damaged human heart have shown the ability to reverse prior damage done to the heart (Williams 2011) and improve cardiac symptoms (Hare 2009). In addition, mesenchymal stem cells have been proposed for treatment in numerous diseases including cancer therapy (Dai 2011), graft vs host disease (GVHD) (Kebriaei 2011), intestinal disease (Manieri 2011), lung injury (Matthay 2010), wound healing (Zebardast 2010) and multiple sclerosis (Uccelli 2011). One of the most attractive aspects of mesenchymal stem cells for clinical application is the ability of these cells to modulate the immune response over a broad range of immunogenic cell types.

Mesenchymal stem cells have been shown to have effects on multiple cell types involved in inflammation and tissue rejection. Preclinical studies have shown that MSCs can evade alloreactive T-cells by the lack of expression of B7-1, B7-2 CD40, and CD-40 ligand, inflammatory co-stimulatory proteins involved in initiating cell-based immune response (Aggarwal 2005). It has been proposed that the effects of MSCs include modulating the response of mature monocyte dendritic cells with the down regulation of TNF-alpha and regulating IL-10 in mature plasmacytoid dendritic cells which reduces the immune-inflammatory response initiated by the two dendritic cell types (Aggarwal 2005; Nauta 2006). In addition, the response of T-cells shifts from a TH1 to a TH2 response with decreases of INF-gamma secretion from TH1 cells and an increase in IL-4 secretion from TH2 cells in the presence of MSC, which further induces TH-2 differentiation inducing a more humoral immunity (TH2) rather than cellular immunity (TH1) (Aggarwal 2005). MSCs also induced an increase in the CD4+ and CD25+ T-regulatory cells (Aggarwal 2005; Zappia 2005; Selmani 2008), which are responsible for creating a more tolerant environment by down regulating the inflammatory T-cell response.

Additionally MSCs can increase healing potential by inducing the expression of IL-6, IL-8, vascular endothelial growth factor and prostoglandin E2 (Aggarwal 2005). All these molecules have been shown to have a role in regeneration in injury models (Rennekampff 2000; Lin 2003; Bao 2009; Yanez 2010). These key features of MSCs make them an attractive cell type to be used with transplanted tissues to promote healing and have resulted in approval of Phase I/II clinical trials using these cells for treating multiple diseases.

Notwithstanding the prior use of MSCs and BMPs in the clinical and pre-clinical applications noted above, their use in the treatment of orthopedic disorders such as fractures and soft tissue damage has been hindered. The need, therefore, exists for compositions and methods of treatment which address the drawbacks of the prior compositions and enable the effective use of BMP-expressing MSCs in the treatment of orthopedic injuries and diseases. The embodiments provided herein fulfill these needs as well as others.

### SUMMARY OF THE INVENTION

In some embodiments, compositions comprising one or more biomaterials, one or more mesenchymal stem cells (MSCs), the MSCs comprising one or more nucleotide sequences encoding one or more bone regeneration proteins, wherein the one or more nucleotide sequences encoding one or more bone regeneration proteins are operably linked to a promoter; and an expression vector nucleotide sequence or a fragment thereof for expressing said bone regeneration protein(s) are provided. In some embodiments, the promoter is constitutive, inducible, or tissue or cell specific. In some embodiments, the one or more biomaterials are selected from the group consisting of collagen, fibrin, silk, agarose, alginate, hyaluronan, chitosan, a polyester such as polylactic-co-glycolic acid, polyethylene glycol, polyethersulfone, a peptide-based biomaterial, a ceramic-based biomaterial, natural tissue including liver, lung, intestinal submucosa (swine) and other tissues which are decelluarized or taken from the patient or subject to be treated and mixtures thereof. In some embodiments, when the composition comprises polyethylene glycol or a derivative thereof the expression vector nucleotide sequence or fragment thereof is not an adenovirus expression vector or an adeno-associated virus expression vector nucleotide sequence or fragment thereof. In some embodiments, the bone regeneration protein is a bone morphogenetic protein (BMP). In some embodiments, the bone regeneration protein is often BMP-2, BMP-4, BMP-5, BMP-7, or any combination thereof.

In some embodiments, the composition is a population of microspheres, a gel, a putty, or a cellular matrix. It is noted that the microspheres may be included in the gel, putty or cellular matrix in preferred aspects of the present invention. In some embodiments, the MSCs are encapsulated by the one or more biomaterials. In some embodiments, the MSCs are not encapsulated by the one or more biomaterials. In some embodiments, the composition is free of encapsulated MSCs.

In some embodiments, the expression vector sequence is a retrovirus, adeno-associated virus, adenovirus, or plasmid expression vector sequence. In some embodiments, the retrovirus is a lentivirus. In some embodiments, the lentivirus is HIV, SIV, or FIV.

In some embodiments, the composition comprises one or more nucleotide sequences encoding SDF-1α, IL-6, IL-8, and/or vascular endothelial growth factor or any combination thereof; and/or a SDF-1α polypeptide, an IL-6 polypeptide, an IL-8 polypeptide, and/or a vascular endothelial growth factor peptide or any combination thereof; and/or prostoglandin E2.

In some embodiments, pharmaceutical compositions comprising any composition described herein are provided. In some embodiments, the composition comprises a pharmaceutically acceptable excipient.

In some embodiments, methods of treating a bone or cartilage disorder are provided. In some embodiments, the methods comprise administering to a subject having the bone or cartilage disorder, one or more of the compositions or pharmaceutical compositions described herein. In some embodiments, the subject is a human, dog, cat, or horse.

In some embodiments, a population of ossification inducing microspheres or a gel, including an in-situ gel which includes a gel capable of polymerizing and/or crosslinking due to temperature or light induction after administration (“polymerizable gel”) are provided. In some embodiments, the microspheres comprise (a) one or more biomaterials selected from the group consisting of collagen, fibrin, silk, agarose, alginate, hyaluronan, chitosan, a biodegradable polyester such as polylactic-co-glycolic acid, polylacic acid, or polyglycolic acid, polyethylene glycol, polyvinylpyrrolidone, polyethersulfone, a peptide-based biomaterial, a ceramic-based biomaterial and mixtures thereof; and (b) one or more mesenchymal stem cells (MSCs) that are encapsulated by and that have been propagated on a scaffolding comprising one or more of the biomaterials, wherein the mesenchymal stem cells (MSCs) which are often cryopreserved:

(1) have been transfected with an adenovirus-based vector comprising a nucleotide sequence which encodes a bone regeneration protein; or
(2) have been transfected with a retrovirus-based vector comprising a nucleotide sequence which encodes a bone regeneration protein; or
(3) have been transfected with a adeno-associated virus-based vector comprising a nucleotide sequence which encodes a bone regeneration protein; or
(4) have been transfected with a plasmid comprising a cDNA which encodes a bone regeneration protein; or
(5) have undergone transposon mutagenesis which introduced into their chromosomes a nucleotide sequence which encodes a bone regeneration protein. In some embodiments, each of the microspheres is homogeneous (i.e. comprise the same components and made in the same manner). In some embodiments, the population of microspheres is heterogeneous. In some embodiments, the population of microspheres may be admixed in the gel, including a polymerizable gel and administered to a patient or subject.

In some embodiments, the bone regeneration protein is bone morphogenetic protein (BMP) selected from the group consisting of heterodimer BMP2/BMP7, BMP-2, BMP-4, BMP-5 and BMP-7.

The mesenchymal stem cells can be autologous (syngeneic) or allogeneic and, optionally, cryopreserved. Ossification-inducing microspheres as described herein may further comprise a cell growth medium and one or more additional cell growth factors.

In some embodiments of ossification-inducing microspheres comprising (a) the biomaterial is polyethylene glycol-diacrylate (e.g., PEG-DA); (b) the bone regeneration protein is selected from the group consisting of heterodimer BMP2/BMP7, BMP-2 and BMP-7; and

(c) the mesenchymal stem cells are cryopreserved.

In some embodiments of the ossification-inducing microspheres, the mesenchymal stem cells are transfected with a lentivirus-based vector comprising a nucleotide sequence which encodes either heterodimer BMP2/BMP7, BMP-2 or BMP-7. In some embodiments, the lentivirus-based vector is either a HIV-based vector or a simian immunodeficiency virus (SIV)-based vector. In some embodiments, the mesenchymal stem cells are transfected with adenovirus type 5 (Ad5BMP2).

In some embodiments, mesenchymal stem cells (MSCs) can be derived from bone marrow, adipose tissue, fetal tissue, peripheral blood or cord (umbilical cord e.g., Wharton's jelly, placental) blood.

In some embodiments of the ossification-inducing microspheres and/or biomaterial which term includes a gel (which includes an in situ gel including a polymerizable gel as described) (a) the mesenchymal stem cells are cryopreserved and (1) have been transfected with an adeno-associated virus (AAV)-based vector comprising a nucleotide sequence which encodes a bone morphogenetic protein, e.g., heterodimer BMP2/BMP7, BMP2 or BMP-7, or (2) have been transfected with a lentiviral-based vector comprising a nucleotide sequence which encodes a bone morphogenetic protein, e.g. heterodimer BMP2/BMP7, BMP2 or BMP-7, or (3) have been transfected with a plasmid comprising a cDNA which encodes a bone morphogenetic protein, e.g., heterodimer BMP2/BMP7, BMP2 or BMP-7; and (b) the biomaterial is polyethylene glycol.

In some embodiments, compositions comprising a population of ossification-inducing microspheres and a population of anti-inflammatory microspheres, (I) wherein each of the ossification-inducing microspheres comprises: (a) one or more biomaterials, preferably selected from the group consisting of collagen, fibrin, silk, agarose, alginate, hyaluronan, chitosan, a biodegradable alkylpolyester such as polylactic acid, polyglycolic acid, polycaprolactone, polylactic-co-glycolic acid, polyethylene glycol, polyethersulfone, polyvinylpyrrolidone, a peptide-based biomaterial, a ceramic-based biomaterial and mixtures thereof; and (b) one or more mesenchymal stem cells (MSCs) that are encapsulated by and that have been propagated on a scaffolding comprising one or more of the biomaterials, wherein the mesenchymal stem cells (MSCs): (1) have been transfected with an adenovirus-based vector comprising a nucleotide sequence which encodes a bone regeneration protein; or (2) have been transfected with a retrovirus-based vector comprising a nucleotide sequence which encodes a bone regeneration protein; or (3) have been transfected with a plasmid comprising a cDNA which encodes a bone regeneration protein; or (4) have undergone transposon mutagenesis which introduced into their chromosomes a nucleotide sequence which encodes a bone regeneration protein; and (II) wherein each of the anti-inflammatory microspheres comprises: (a) one or more biomaterials selected from the group consisting of collagen, fibrin, silk, agarose, alginate, hyaluronan, chitosan, a biodegradable polymer such as polylactic acid, polyglycolic acid, polylactic-co-glycolic acid, polyethylene glycol, polyethersulfone, polyvinylpyrrolidone, a peptide-based biomaterial, a ceramic-based biomaterial and mixtures thereof; and (b) one or more mesenchymal stem cells (MSCs) that are encapsulated by and that have been propagated on a scaffolding comprising one or more of the biomaterials, wherein the cryopreserved mesenchymal stem cells (MSCs): (1) have been transfected with an adenovirus-based vector comprising a nucleotide sequence which encodes SDF-1α; or (2) have been transfected with a retrovirus-based vector comprising a nucleotide sequence which encodes SDF-1α; or (c) have been transfected with a plasmid comprising a cDNA which encodes SDF-1α; or (d) have undergone transposon mutagenesis which introduced into their chromosomes a nucleotide sequence which encodes SDF-1α; wherein the types of biomaterials and mesenchymal stem cells which comprise the ossification-inducing microspheres and the anti-inflammatory microspheres may be the same or different are provided.

Pharmaceutical compositions comprising a plurality of ossification-inducing microspheres or any composition described herein are provided. In some embodiments, the pharmaceutical compositions comprise a pharmaceutically acceptable excipient. In some embodiments, the pharmaceutical composition is injected or implanted at a site of bone or tissue injury, disorder, or disease. In some embodiments, the pharmaceutical composition is a transdermal composition.

In other embodiments, methods of treating a subject suffering from, or at risk of developing, one or more disorders selected from the group consisting of bone fracture, long bone nonunion, orthopedic soft tissue injury, spinal injury (which term includes a severed spine), skeletal and cartilage deficits, bone damage associated with primary bone cancers (e.g. osteocarcinoma), congenital bone malformation or nonunion, alveolar bone defects, cranial bone defects, facial bone defects, short bone defects, flat bone defects, irregular bone defects, sesamoid bone defects, cartilage defects, dentoalveolar defects, connective tissue defects, and collagen membrane defects are provided. In some embodiments, the method comprises administering to the subject at a therapeutically appropriate site a pharmaceutically effective amount of ossification-inducing microspheres or any composition described herein.

In some embodiments, the methods promote osteoblast adhesion, growth and differentiation, and allow vascular in-growth and bone-tissue formation.

In other aspects, compositions and methods described herein are used in conjunction with synthetic bone substitutes, such as hyaluronic acid (HA), β-tricalcium phosphate (β-TCP), calcium-phosphate cements and glass ceramics. These additional synthetic bone substitutes are optionally used with compositions described herein as adjuncts or alternatives to autologous bone grafts, as they promote the migration, proliferation and differentiation of bone cells for bone regeneration. Such adjuvants can be used in the regeneration of large bone defects, where the requirements for grafting material are substantial, and these synthetics can be used in combination with autologous bone graft, growth factors or cells. Non-biological osteoconductive substrates, such as fabricated biocompatible metals (for example, porous tantalum) that offer the potential for absolute control of the final structure without any immunogenicity can also be used. See Dimitriou, et al., “Bone regeneration: current concepts and future directions”, BMC Medicine 2011, 9:66.

Processes of making a population of ossification-inducing microspheres as described herein are also provided.

Mesenchymal stem cells are also provided. In some embodiments, the MSCs have been

(a) transfected with an adenovirus-based vector comprising a nucleotide sequence which encodes a bone regeneration protein, especially a bone morphogenetic protein; (b) transfected with a retrovirus-based vector comprising a nucleotide sequence which encodes a bone regeneration protein, especially a bone morphogenetic protein; (c) transfected with a plasmid comprising a cDNA which encodes a bone regeneration protein, especially a bone morphogenetic protein; or (d) subjected to transposon mutagenesis which introduced into the cell's chromosomes a nucleotide sequence which encodes a bone regeneration protein, especially a bone morphogenetic protein. In each instance, the nucleotide sequence is capable of expressing the encoded bone morphogenetic protein.

In some embodiments, non-encapsulated mesenchymal stem cells are used to treat a variety of bone (e.g. orthopedic) or cartilage disorders as described herein.

### BRIEF DESCRIPTION OF THE FIGURES

FIG. 1. Sheep 30 days radiograph as described in Example 2. Left radiograph (LH). Proximal tibial defect without bone formation (yellow arrow pointing at drill hole through cortex). Right radiograph (R). Bone formation (yellow arrow pointing to new bone formed on the surface of the bone below the linear defect in the cortex).

FIG. 2. Sheep 30 days radiograph as described in Example 2. Left radiograph (LH). Proximal tibial defect without bone formation (yellow arrow pointing at drill hole through cortex). Right radiograph (R H). Bone formation (yellow arrow pointing to new bone formed on the surface of the bone below the linear defect in the cortex).

FIG. 3. Sheep 30 days CT as described in Example 2. CAT scan from the R tibia shown in FIG. 2. Note adequate osseous integration with the cortex as seen in the center image.

FIG. 4. As determined in the experiment of Example 2, no bone formation was observed in the non-cyclosporine animal (6110) despite treatment of ulna and tibia with BMP-2-MSCs.

FIG. 5.1. Characterization of ovine MSCs and proliferation and BMP-2 transduction of ovine and porcine MSCs as determined in the experiment of Example 1. Ovine MSCs A) osteogenic differentiation is seen through dark staining of calcium depositions by Von Kossa silver nitrate staining. B) condrogenic differentiation with chondroitin sulfate proteoglycans stained blue with Alcian Blue staining, and C) adipogenic differentiation as seen through intracellular lipid staining with Oil Red O. D) Ovine A, ovine B, and porcine cell line proliferation rates E) Proliferation rates of ovine A and ovine B following transduction with 15,000 vp/cell of adenoviral BMP-2 F) Transduction with 15,000 vp/cell adenoviral BMP-2 production from ovine and porcine MSCs both with and without cryopreservation.

Supplemental FIG. 5.1. Transductions of cells at 36,000 cells/cm² were optimized with various amounts of vp/cell as determined in the experiment of Example 1. 5,000; 75,000; 10.000; and 15,000 vp/cell were used in transductions. Media samples were quantified at 24 hours with a BMP-2 ELISA. (*) represent statisitcal difference from other samples (p<0.05).

FIG. 5.2. Viability of cells on day 0 and 4 post encapsulation with and without cryopreservation as determined in the experiment of Example 1. Cells were stained with calcien AM for live (green) and ethidium homodimer for dead (red). On the day of encapsulation ovine A MSCs stained for A) Live, B) Dead and C) overlay. And on day 4 post encapsulation E) ovine A MSCs stained for F) Live, H) Dead and G) overlay. D) Graphical representation of counts of 90 images. Cryopreserved ovine A encapsulated MSCs on day of thaw I) Live, J) Dead and K) overlay. Day 4 post thaw ovine A MSCs stained for L) Live, M) Dead and N) overlay. H) Graphical representation of counts of 90 images (averaging 87,000 cells per image). (*p<0.0001).

FIG. 5.3. BMP-2 production in microencapsulated MSCs as determined in the experiment of Example 1. A) Cells were transduced with 15,000 vp/cell adenoviral BMP-2 prior to encapsulation and plated out freshly or cryopreserved. (+,# p<0.01), B) Ovine A and ovine B cryopreservation and BMP-2 transduction effect on MSC viability on day of encapsulation (day of thaw for cryopreserved samples). And C) 4 days after encapsulation (p<0.05).

FIG. 5.4. Structural analysis of freshly prepared and cryopreserved encapsulated MSCs as determined in the experiment of Example 1. A) Phase contrast of encapsulated ovine A MSCs showed clear borders on microbeads. B) SEM of MSC microbeads showed a uniform surface. C) SEM of MSC microbeads of all sizes showed uniform structure. D) Phase contrast of cryopreserved encapsulated MSCs did not show appreciable damage E) SEM of cryopreserved MSC microbeads showed a uniform surface. F) SEM of cryopreserved encapsulated MSCs showed no damage to beads of various sizes.

FIG. 5.5. BMP2-transducedmicroencapsulated MSCs bone formation in a mouse model for heterotopic ossification. 3×10⁶ ovine BMSCs transduced with 15,000 vp/cell were injected into the hind limb of a NOD/SCID mouse. The resulting heterotopic ossification was observed by X-ray and MicroCT for (a) and (b) for freshly prepared BMP2 microencapsulated MSCs and (d) and (e) for cryopreserved BMP2 microencapsulated MSCs. (c) The volume of the resulting heterotopic ossification was not different between the two groups.

FIG. 6. As determined in the experiment of Example 2, one sheep (6184) out of 4 treated with cyclosporine showed bone deposition within the ulna defect following injection with BMP-2-MSCs. This was inferior to that observed with cancellous bone grafting.

FIG. 7 shows the transduction efficiency of pR-EF1a-TagRFP-2A-Puro Cellecta lentivirus in ovine MSC. MSCs were transduced at 26,109 cells/cm² at 0 (FIG. 7 A, B, G, H, M, N), 10 (FIG. 7. C, D, I, J, O, P), and 50 MOI (FIG. 1. E, F, K, L, Q, R) with lentivirus and 5 μg/mL hexadimethrine bromide, and phase contrast and fluorescent images were taken at 24 (FIG. 7 A-F), 48 (FIG. 1, G-L), and 72 (FIG. 7, M-R) hours post transduction (100×).

FIG. 8 shows sheep MSCs transduced with BMP2. Sheep bone marrow derived MSCs were transduced with pR-EF1a-BMP2 at multiplicities of infection 0, or 10, as indicated (FIG. 8). Secreted BMP-2 was measured from tissue culture supernatants using a well characterized enzyme-linked immunosorbent assay (ELISA) procedure (R & D Systems), at timepoints 48, 72, and 96 hours following transduction. Secreted BMP2 was assayed in triplicate with median values shown. At 48 hours post-transduction, MSCs produced approximately 19,000 pg/ml BMP2. Levels of BMP2 increased to 103,000 pg/ml at 72 hours, and 153,000 pg/ml at 96 hours following transduction.

FIG. 9 shows that human bone marrow mesenchymal stem cells MSCs secrete Increasing amounts of BMP2 post-transduction. Umbilical (Wharton's Jelly) derived MSCs were transduced with pR-EF1a-BMP2 at multiplicities of infection 0, 10, or 50 as indicated (FIG. 9). Secreted BMP-2 was measured from tissue culture supernatants using a well characterized enzyme-linked immunosorbent assay (ELISA) procedure (R & D Systems), at timepoints 48, 72, 96, and 120 hours following transduction. Amounts of BMP2 increased from approximately 0.2 pg/cell at 10 MOI 24 hours post-transduction, to 1.5-1.8 pg/cell secreted with 10 viral particles per cell or 50 viral particles per cell respectively.

FIG. 10 shows that human umbilical MSCs secrete greater amounts of BMP2 post-transduction. Umbilical (Wharton's Jelly) derived MSCs were transduced with pR-EF1a-BMP2 at multiplicities of infection 0, 10, or 50 as indicated (FIG. 10). Secreted BMP-2 was measured from tissue culture supernatants using a well characterized enzyme-linked immunosorbent assay (ELISA) procedure (R & D Systems), at timepoints 48, 72, 96, and 120 hours following transduction. While initial timepoints produced comparable levels of BMB2 as bone marrow derived MSCs, by 72 hours umbilical cells were producing 1.5 to 2 pg/cell, approximately the same levels produced by bone marrow derived stem cells at 120 hours. The amount of secreted BMP2 continued to increase with time and MOI at levels that were fairly consistent between experiments (red and green bars represent separate transductions). Maximum levels of BMP2 measured at 120 hours post-transduction exceeded 4 pg/cell.

FIG. 11 shows a comparison of BMP2 produced from adeno vs lenti virus. BMP2 production was measured from monolayers using adenoviral and lentiviral constructs in ovine bone marrow MSC, human umbilical MSC, and human bone marrow MSC (FIG. 11). Cells were transduced at 15,000 MOI (adenoviral; ovine MSC), 10 MOI (lentiviral; ovine and human MSC), and 50 MOI (lentiviral; human MSC). Media was harvested from transduced cells at 48, 72, 96, and 120 hours post transduction (except for ovine MSC, no 120 hour sample) and frozen at −80° C. BMP2 was quantified from harvested media using a BMP2 ELISA kit.

FIG. 12 shows a site map for lentiviral vector pR-EF1-BMP2.

FIG. 13 shows that lentiviral BMP2 expressing MSCs when administered to laboratory test animals formed bone in vivo. Experiments described in detail herein showed that mice formed bone as early as 7 days post injection.

### DETAILED DESCRIPTION

Unless otherwise noted, the terms used herein are to be understood according to conventional usage by those of ordinary skill in the relevant art.

It is to be understood that as used in the specification and in the claims, “a” or “an” can mean one or more, depending upon the context in which it is used. Thus, for example, reference to “a cell” can mean that at least one cell can be utilized.

As used in this document, terms “comprise,” “have,” and “include” and their conjugates, as used herein, mean “including but not limited to.” While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

The compositions and methods described herein may be understood more readily by reference to the description contained herein and the Examples included herein. However, before the present compositions and methods are disclosed and described, it is to be understood that the embodiments are not limited to specific conditions, or specific methods, etc., as such may, of course, vary, and the numerous modifications and variations therein will be apparent to those skilled in the art.

Standard techniques for growing cells, separating cells, and where relevant, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The term “subject”, or in some instances, “patient” is used throughout the specification within context to describe an animal. The terms “subject” and “patient” can be used interchangeably. The animal can be human or non-human. In some embodiments, the subject is what is commonly referred to as a veterinary animal or a domesticated animal. Examples of a veterinary animal include, but are not limited to, dogs, cats, pigs, horses, cows, birds, and the like. The subject can also be a non-human primate, such as, but not limited to, monkey or chimpanzee.

As used herein, the term “expression vector” refers to a composition that can be used to express a protein or nucleotide sequence of interest. Examples of expression vectors include, but are not limited to, plasmids and virus based expression systems. The virus based expression system can be a retrovirus, adenovirus, or adeno-associated virus expression system. Where a virus expression system is used, in some embodiments, the virus infects the target cell, and the protein of interest is expressed from nucleotide sequences encoding the protein of interest. The nucleotide sequence is normally part of the virus's genome (e.g. DNA or RNA). In some embodiments, the nucleotide sequence encoding a bone regeneration protein or other protein of interest is integrated into the cell's genome. In some embodiments, the sequence is maintained extrachromosomally.

The compositions and methods described herein utilize the functions and properties of proteins that facilitate or enhance the growth of bone. This can also be referred to as “bone regeneration.” Some of these proteins are referred to as bone growth factors or as bone morphogenetic proteins. Examples of these proteins include, but are not limited to, lactoferrin, bone morphogenetic protein 2 (BMP2), BMP3, BMP4, BMP5, BMP6, BMP7, BMP8a and BMP9. In most embodiments, BMP2, BMP4, BMP5 and/or BMP 7 are used. In some embodiments, a bone regeneration protein is selected from the group consisting of BMP2 and BMP7. In some embodiments, the regeneration protein is a heterodimer of BMP2/BMP7. In some embodiments, the bone regeneration protein is not a heterodimer, but is a homodimer. In some embodiments, the bone regeneration is a heterodimer

In addition to the proteins described herein, all isoforms, variants, homologs, derivatives and peptides of the bone morphogenetic protein family or other sequences or proteins described herein are included. In some embodiments, the variants (e.g. fragments or analogs) of bone morphogenetic proteins have at least or about 85%, 90%, 91%, 92%, 93%, 94%, 95%, 96%, 97%, 98%, or 99% sequence identity as compared to the native, or naturally occurring proteins. These variants although not identical in sequence retain the function of the native proteins. In some embodiments, the variants have at least or about 85%, 90%, 91%, 92%, 93%, 94%, 95%, 96%, 97%, 98%, or 99% sequence homology. Variants can also include sequences with conservative substitutions at the amino acid level or silent substitutions at the nucleotide sequence level that do not affect the amino acid sequence of the protein encoded by the nucleotide sequence. It is well understood that nucleic acid sequences can be changed without changing the protein that is encoded for due to the degeneracy of the genetic code. Nucleic acid sequences encoding the proteins described herein are also included and used in the compositions and methods described herein.

Representative nucleotide and amino acid sequences which encode or comprise illustrative bone regeneration proteins, including nucleotide and amino acid sequences which encode or comprise heterodimer BMP2/BMP7, BMP2 or BMP-7, as well as useful vectors, are provided, for example, in U.S. Patent Application Document No. 20090105137 (Ser. No. 11/930,115), the complete contents of which are hereby incorporated by reference. Sequence information for heterodimer BMP2/BMP7, BMP2 and BMP-7 and other bone regeneration proteins are also available from a number of sources, including the NIH GenBank and Entrez Gene and coremine corn websites. Those of ordinary skill in the art will be able to identify additional useful sequences that are complementary to or which hybridize with the bone regeneration protein nucleotide sequences identified in the prior art. A sequence of BMP2 can also be found at GenBank Accession No. NM_{—}001200.2, which is hereby incorporated by reference. A nucleotide sequence encoding BMP2 is also described herein. The protein sequence can also be found as described herein and appended hereto. In some embodiments, BMP-2 can be referred to as BMP2A. In some embodiments, the nucleotide sequence comprises the coding sequence for BMP2. In some embodiments, the nucleotide sequence comprises nucleotides 786-1976 of the sequence described herein. The bone regeneration protein may be from the same species as the subject or from a different species, although in certain embodiments it may be preferred that the bone regeneration protein be from the same species.

A sequence of BMP7 can also be found at GenBank Accession No. NM_{—}001719.2, which is hereby incorporated by reference. A nucleotide sequence encoding BMP7 is also described herein and appended hereto. The protein sequence can also be found as described herein and appended hereto. In some embodiments, BMP-7 can be referred to as OP-1. In some embodiments, the nucleotide sequence comprises nucleotides 530-1825 of the sequence described herein.

As discussed above, for different species, such as canine, etc., the specific species protein and nucleotide sequence encoding the same can be used and may be preferable. For example, for canine, a canine bone morphogenetic protein can be used. In some embodiments, the canine protein is canine BMP-2. In some embodiments, the protein sequence comprises the sequence appended hereto. In some embodiments, the nucleotide sequence comprises the sequence appended hereto. In some embodiments, the sequence is found at Accession No. XP_{—}534351.2 or at XM_{—}534351.3, both of which are hereby incorporated by reference in its entirety.

Throughout the present specification, the gene and gene product SDF-1 is described. SDF-1 can also referred to as CXCL12, IRH; PBSF; SDF1; TLSF; SDF1A; SDF1B; TPAR1; SCYB12. SDF-1 can also produce another product referred to as SDF-1α. SDF-1B can also be used in place or in conjunction with SDF-1α. The sequence of SDF-1α can be found, for example at Accession No. L36033. SDF-1α is also described, for example, in BLOOD, 1 Apr. 2004, VOLUME 103, NUMBER 7, 2452-2459, which is hereby incorporated by reference in its entirety. Variants of SDF-1α can also be used. The sequence of SDF-1α is known and accessible.

The term “homology,” as used herein, refers to a degree of complementarity. There may be partial homology or complete homology. The word “identity” may substitute for the word “homology.”

The phrases “percent homology,” “% homology,” “percent identity,” or “% identity” refer to the percentage of sequence similarity found in a comparison of two or more amino acid or nucleic acid sequences. Percent identity can be determined electronically, e.g., by using the MEGALIGN program (LASERGENE software package, DNASTAR). The MEGALIGN program can create alignments between two or more sequences according to different methods, e.g., the Clustal Method. (Higgins, D. G. and P. M. Sharp (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be calculated by the Clustal Method, or by other methods known in the art, such as the Jotun Hein Method. (See, e.g., Hein, J. (1990) Methods Enzymol. 183:626-645.) Identity between sequences can also be determined by other methods known in the art, e.g., by varying hybridization conditions. Other alignment tools can also be used. For example at the National Center for Biotechnology Information (NCBI) website, one can use a Basic Local Alignment Search Tool (BLAST) to determine the percent homology or identity of two sequences. The alignment can be done, for example, with default settings.

The term “effective amount” is used throughout the specification to describe concentrations or amounts of components such as mesenchymal stem cells, components of cell media or other agents, including biomaterials that are effective for producing an intended result within the context of using one or more of the compositions and/or methods described herein. Effective amounts are those that are generally known to those of ordinary skill in the art and are typically used when growing mesenchymal stem cells, modifying those cells and administering them to a patient for therapeutic purposes and as otherwise described herein.

The term “cryopreserved” refers to a cell or population of cells that has been cryopreserved. Cells can be cryopreserved using any method. For example, the cell or population of cells can be cryopreserved in the presence of one or more cryoprotectants. A cryoprotectant is a compound or composition that protects the cells when frozen to maintain the viability of the cell when it is thawed. Examples of cryoprotectants include, but are not limited to, dimethylsulfoxide (DMSO), acetamide, dimethylacetamide, ethylene glycol, propylene glycol, glycerol, and the like. The cryoprotectants can be added to a mixture of cells to be cryopreserved, for example, in order to limit cell damage principally during the cryopreservation step(s). Mesenchymal stem cells which have been modified to express a bone morphogenetic protein as otherwise described herein may be cryopreserved as an optional step to the methods otherwise disclosed herein. The cryopreservation methods are standard practice and well known in the art. The cells can then be thawed prior to being used.

The terms “transfect”, “transfecting”, “transduct”, “transduction” are used (in many instances synonymously) to describe a process of introducing nucleic acids into cells. The term transfecting is used notably for introducing non-viral DNA (generally plasmids, although naked DNA, including supercoiled naked DNA and RNA including modified mRNA and MicroRNA may also be used) into eukaryotic cells, but the term may also refer to other methods and cell types, although other terms may also be used. Transfection or transduction of animal cells typically involves opening transient pores or “holes” in the cell membrane, to allow the uptake of material into the cells to be transfected. Transfection can be carried out using an agent such as calcium phosphate or other agent to assist transfection into the target cell, by electroporation, or by mixing a cationic lipid with the material to produce liposomes, which allow the nucleic acid sequence to enter the cell. Transduction is a term which describes the process by which foreign DNA is introduced or transferred from one bacterium to another by a virus. This term also refers to the process whereby foreign DNA is introduced into another cell via a viral vector. Transduction does not require cell-to-cell contact (which occurs in conjugation), and it is DNAase resistant, but it often is benefitted by the inclusion of a transduction factor such as GeneJammer™ or TransDux™ transduction reagents. Transduction is a relatively common tool used by those of skill to stably introduce or integrate a foreign gene into a host cell's genome. Nucleic acid sequences can also be introduced into a cell through infection with a virus. The virus can, for example, infect a cell and then express a nucleic acid sequence of interest. The nucleic acid sequence can encode one or more of the proteins, or variants thereof, described herein.

The term “somatic cell” is used to describe any cell which forms the body of a multicellular organism that is other than a gamete, germ cell, gametocytes or undifferentiated stem cell. In contrast to somatic cells, gametes are cells that are involved in sexual reproduction, germ cells are gamete precursory cells and stem cells are cells that can divide (mitosis) and differentiate into a variety of cell types. Somatic cells are diploid. In some embodiments, any somatic cell may be used to induce pluripotent stem cells, including mesenchymal stem cells. Examples include, but are not limited to, those cells which may be readily propagated, especially including fibroblast cells, including adult and embryonic fibroblast cells. Other types of cells that can be used include, but are not limited to, stomach cells, liver cells, keratinocytes, amniotic cells, blood cells, adipose cells, neural cells, melanocytes, among numerous others, may also be used. Stem cells from other tissues can also be used. For example, stem cells isolated from adipose tissue can be used.

In some embodiments, hematopoietic stem cells and mesenchymal stem cells (MSCs) can be used. Hematopoietic stem cells, or blood precursor stem cells, have become common place in hospitals for the treatment of blood and immunodeficiency disorders. Mesenchymal stem cells are most often used and represent the preferred stem cell for use in the present invention.

Mesenchymal stem cells are multipotent cells which are capable of differentiating into adipocytes, osteocytes, chondrocytes, myocytes, neurocytes, cardiomyocytes, etc. These cells can also function to regulating immune responses. The mesenchymal stem cells can be isolated and cultured from various tissues, but their capacity and cell surface markers are different from one another depending on the origins thereof. Examples of mesenchymal stem cells are generally defined by cells which can differentiate into osteocytes, chondrocytes and myocytes; have a spiral form; and are CD73(+), CD105(+), CD34(−), and CD45(−), which are basic cell surface markers. Combining the lineage differentiation capacity of MSCs to form bone, cartilage and adipose, with genetic engineering can be provided with the MSCs described herein and methods of using the same provide new therapeutics for treating skeletal and cartilage deficits. Mesenchymal stem cells (MSCs) as described herein satisfy the criteria specified in Dominici, et al. “Minimal criteria for defining multipotent mesenchymal stromal cells. The International Society for Cellular Therapy position statement”, Cytotherapy (2006) Vol. 8, No. 4, 315-317, the complete contents of which are hereby incorporated by reference. Mesenchymal stem cells for use in the present invention may be obtained from any source, including peripheral blood, fallopian tube, and fetal liver and lung, but principal sources include bone marrow and umbilical cord/placental blood. It is noted that the use of mesenchymal stem cells which are isolated from umbilical cord/placental blood unexpectedly provide substantially greater expression of bone regeneration proteins, especially including bone morphogenetic proteins (BMP), than do mesenchymal stem cells from other sources, including MSCs which are isolated from bone marrow. Thus, the use of mesenchymal stem cells (MSCs) which are isolated from umbilical cord/placental blood provide unexpectedly favorable results when used in the present invention, including substantially better results than when mesenchymal stem cells isolated from other sources (especially including bone marrow) are used in the present invention. Thus, the use of MSCs isolated from umbilical cord/placental blood samples are often used in the present invention and are preferred. MSCs isolated from bone marrow or umbilical cord/placental blood samples may be obtained commercially from a number of sources, including Fisher Scientific, Inc., (USA), PromoCell (Germany), R&D Systems, Inc. (USA), StemCell Technologies, Inc. (Canada), among others.

In some embodiments, the mesenchymal stem cells (MSCs) may be transfected or infected with a virus based vector. For example, an adenovirus-based vector comprising a nucleotide sequence which encodes a bone regeneration protein can be used. In some embodiments, the MSCs are transfected or infected with a retrovirus-based vector comprising a nucleotide sequence which encodes a bone regeneration protein. In some embodiments, the cells are transfected with a plasmid comprising a cDNA which encodes a bone regeneration protein, especially including a bone morphogenetic protein (BMP) as otherwise described herein. The cells can be also be subjected to transposon mutagenesis. Transposon mutagenesis introduces into the cell's chromosomes a nucleotide sequence that encodes a bone regeneration protein. The virus based vector can also comprise nucleotide sequences encoding one or more proteins described herein.

Non-limiting examples of transfection techniques are illustrated in Olmsted, et al., “Adenovirus-Mediated BMP Expression in Human Bone Marrow Stromal Cells”, Journal of Cellular Biochemistry 82:11-21 (2001), the complete contents of which are hereby incorporated by reference. Other useful techniques are well-known to those of ordinary skill in the art. The two major classes of methods are those that use recombinant viruses (sometimes called biological nanoparticles or viral vectors) and those that use naked DNA or DNA complexes (non-viral methods). A purely illustrative, and non-limiting, technique is described in the following paragraph.

For example, an adenovirus can be used to express human BMP2 or other bone morphogenetic protein. For example, a replication defective human type 5 adenovirus (Ad5) containing a cDNA for BMP2 in the E1 region of the virus can be constructed by in vivo homologous recombination in 293 cells which constitutively produces E1 proteins. The human BMP2 cDNA can be constructed by reverse transcription polymerase chain reaction using high fidelity Pfu Turbo DNA polymerase (Stratagene, La Jolla, Calif.). The BMP2 clone can be cloned into an adenovirus transfer plasmid, pCA14 (Microbix, Toronto, Canada) which has map units 0±1.4 and 9±16 of human adenovirus 5 and a deletion in E1 (1.4±9 map units). BMP2 can be cloned into the E1 deleted region which may, for example, contain an upstream human cytomegalovirus promoter, SV40 enhancer, and downstream SV40 polyadenylation sequence. Other promoters can also be used to drive the expression of the BMP2 protein. Other types of promoters are described herein. The vector can then be co-transfected by calcium phosphate precipitation (Promega, Madison, Wis.) along with the ClaI fragment of adenovirus DNA that contains a green fluorescent protein (GFP) marker gene as described by Davis et al., 1998. The appearance of clear plaques demonstrates recombination and several can be selected for further plaque purification and screening, as described by Davis et al., 1998. Viral DNA can be isolated [Davis et al., 1998] and viral lysates with the correct DNA structure can then be tested for production of BMP2 protein by infection of A549 and cell extracts can be immunoblotted as described herein. In some embodiments, a control virus can be similarly constructed which contained a green fluorescent protein expression cassette in the E1 region.

The virus can then be propagated and/or purified. For example, viral lysates that are positive for BMP2 protein expression can be expanded by infection of, for example, 293 cells as described in Davis et al., 1996. A crude lysate can be generated by three cycles of freeze-thawing and then cellular debris can be pelleted by centrifugation. This lysate can be used to infect 293 cells. In some embodiments, at maximal cytopathic effect the virus can be harvested and again subjected to three cycles of freeze thawing. In some embodiments, the virus can then banded on a series of two cesium chloride gradients [Davis et al., 1996] and then desalted using an Econo-Pac1 10 DG disposable size exclusion column (Bio-Rad Laboratories, Hercules, Calif.). This procedure can be modified depending on the protein to be expressed as well as the vector to be used. Other vectors can also be used and modified according to the type of vector and the desired result.

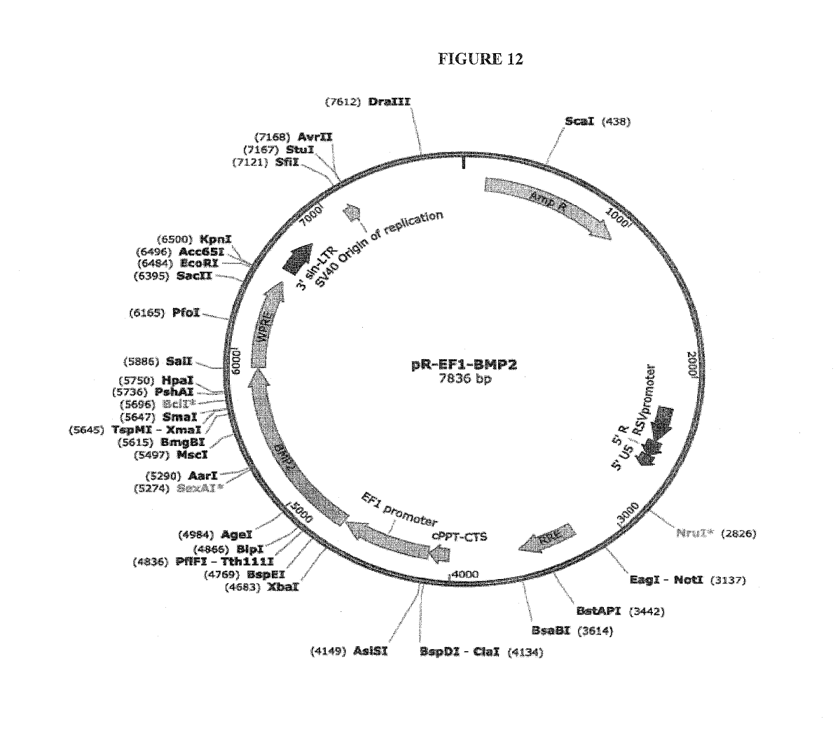

In some embodiments, one or more of the following methods can be used to introduce bone regeneration protein genes into cells for expression of the bone regeneration protein. Examples include, but are not limited to, plasmid based. cDNA is introduced through breaks in DNA; transposons based that utilizes transposon mutagenesis, or transposition mutagenesis, which is a biological process that allows genes to be transferred to a host organism's chromosome; retrovirus; adenovirus, or adeno-associated virus (AAV). In some embodiments, the retrovirus is a lentivirus. Lentiviruses insert their genome into the infected cell's genome following infection resulting in stable long-term gene expression. Lentiviruses have the ability to infect both dividing and non-dividing cells. Thus, in some embodiments, lentiviral-based vectors can be used to deliver genes into non-dividing human cells. Examples of lentiviruses include, but are not limited to HIV or simian immunodeficiency virus (SIV). Preferred lentiviral-based vectors are based on HIV, one of which is set forth in attached FIG. 12 and used in the examples described herein. Among other HIV lentiviral vectors are those described in Allies and Naldini, “Lentiviral Vectors”, Current Topics in Microbiology and Immunology Volume 261, 2002, pp 31-52 and references cited therein. Vectors using AAV can infect both dividing and non-dividing cells and persist in an extrachromosomal state without integrating into the genome of the host cell. Another example of a lentivirus expression vector or system is described in U.S. Pat. No. 6,712,612, which is hereby incorporated by reference in its entirety. In some embodiments, the expression vector is an SIV expression vector that comprise a. an SIV 5′ LTR, or a modified version thereof in which all or part of the U3 region of the 5′ LTR is replaced by a non-SIV promoter, or a derivative thereof; b. an SIV packaging sequence or a derivative thereof; c. an SIV rev-response element or a derivative thereof; d. one of a gene or a cloning site; and e. one of a 3′ SIV LTR, a modified version thereof in which a U3 region is inactivated, or a derivative thereof.

In general, any method used to deliver a nucleotide sequence to a cell can also be used in vitro to deliver bone regeneration protein genes into MSCs for expression of bone regeneration by that cell. Lentiviruses are often used. Biological nanoparticles, naked DNA or DNA complexes (non-viral methods) are all suitable. Injection of naked DNA, electroporation, the gene gun, sonoporation, magnetofection, and the use of oligonucleotides, lipoplexes, dendrimers, and inorganic nanoparticles are other examples of methods that can be used to deliver bone regeneration protein genes, especially bone morphogenetic protein (BMP) genes into MSCs for administration to a subject and eventual expression of bone regeneration protein at the site of activity.

The term “multiplicity of infection” or MOI refers to the ratio of infectious agents (e.g, a phage or virus) to infection targets (e.g. MSCs). Pursuant to the present invention, when a group of cells is inoculated with infectious virus particles, the multiplicity of infection or MOI is the ratio of the number of infectious virus particles to the number of target cells present in a defined space.

As used herein, the terms “nucleotide” or “nucleic acid” refer to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Thus, “nucleotide” is used as recognized in the art to include those with natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, e.g., Usman and McSwiggen, supra; Eckstein, et al., International PCT Publication No. WO 92/07065; Usman et al, International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include, hypoxanthine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine and uracil at 1′ position or their equivalents.

By “complementary” or “complementarity” is meant that a nucleic acid can form hydrogen bond(s) with another nucleic acid sequence by either traditional Watson-Crick or Hoogsteen base pairing. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary, respectively). To determine that a percent complementarity is of at least a certain percentage, the percentage of contiguous residues in a nucleic acid molecule that can form hydrogen bonds (e.g., Watson-Crick base pairing) with a second nucleic acid sequence is calculated and rounded to the nearest whole number (e.g., 12, 13, 14, 15, 16, or 17 nucleotides out of a total of 23 nucleotides in the first oligonucleotide being based paired to a second nucleic acid sequence having 23 nucleotides represents 52%, 57%, 61%, 65%, 70%, and 74%, respectively; and has at least 50%, 50%, 60%, 60%, 70%, and 70% complementarity, respectively). As used herein, “substantially complementary” refers to complementarity between the strands such that they are capable of hybridizing under biological conditions. Substantially complementary sequences have 60%, 70%, 80%, 90%, 95%, or even 100% complementarity. Additionally, techniques to determine if two strands are capable of hybridizing under biological conditions by examining their nucleotide sequences are well known in the art.

Single-stranded nucleic acids that base pair over a number of bases are said to “hybridize.” Hybridization is typically determined under physiological or biologically relevant conditions (e.g., intracellular: pH 7.2, 140 mM potassium ion; extracellular pH 7.4, 145 mM sodium ion). Hybridization conditions generally contain a monovalent cation and biologically acceptable buffer and may or may not contain a divalent cation, complex anions, e.g. gluconate from potassium gluconate, uncharged species such as sucrose, and inert polymers to reduce the activity of water in the sample, e.g. PEG. Such conditions include conditions under which base pairs can form.

Useful transposon mutagenesis techniques are summarized in Ivics, et al., “The expanding universe of transposon technologies for gene and cell engineering”, DNA 2010, 1:25, which is hereby incorporated by reference.

Representative useful biomaterials, cellular propagation conditions and methods, biomaterial scaffold design and encapsulation techniques are described herein and are also disclosed in references such as Willerth, S. M. and Sakiyama-Elbert, S. E., Combining stem cells and biomaterial scaffolds for constructing tissues and cell delivery (Jul. 9, 2008), StemBook, ed. The Stem Cell Research Community, StemBook, doi/10.3824/stembook.1.1.1, http://www.stembook.org., the complete contents of which are hereby incorporated by reference.

As used herein, a “scaffold” is a structure, platform or framework capable of supporting three-dimensional tissue formation. The scaffold can be artificial, fabricated, or made from naturally occurring materials. For example, a scaffold can allow the ingrowth of cells and/or extracellular matrix., within or upon the surface of the scaffold. The scaffold can also allow the formation of a tissue that can be then grafted or placed in contact with a bone or cartilage defect or disorder. A scaffold can also contain bioactive agents and also be able to release the bioactive agents to the environment where the scaffold is placed. In some embodiments, the scaffold is an artificial fabricated structure for the delivery of bioactive agents. The scaffold can be a temporary structure or a permanent structure. In some embodiments, the bioactive agent is a MSC or other composition described herein.

Examples of scaffold biomaterials upon which mesenchymal stem cells, or other cells, can be grown, or within which mesenchymal stem cells, or other types of cells, can be encapsulated (often in microcapsules) include, but are not limited to glycosaminoglycan, silk, fibrin, a gelatinous support protein matrix such as MATRIGEL®, among others, animal decelluarized tissue, for example lung, liver or swine intestinal submucosa or tissue which is obtained from the patient or subject to be treated, peptide hydrogel, poly-ethyleneglycol (PEG), polyethylene glycol diacrylate (PEG-DA), polyhydroxy ethyl methacrylate, polyvinyl alcohol, polyacrylamide, poly(N-vinyl pyrolidone) (polyvinylpyrrolidone), poly lactic acid (PLA), poly glycolic acid (PGA), poly lactic-co-glycolic acid (PLGA), poly e-carpolactone (PCL), polyethylene oxide, poly propylene fumarate (PPF), poly acrylic acid (PAA), hydrolysed polyacrylonitrile, polymethacrylic acid, polyethylene amine, alginic acid, pectinic acid, carboxy methyl cellulose and other cellulosics, hyaluronic acid, heparin, heparin sulfate, chitosan, carboxymethyl chitosan, chitin, pullulan, gellan, xanthan, collagen, gelatin, carboxymethyl starch, carboxymethyl dextran, chondroitin sulfate, in-situ gels such as form-fitting gels, including in-situ polymerizable gels, natural tissue, including lung, liver and mucosal tissue from the patient or subject to be treated or decellularized tissue from an animals such as swine intestinal mucosal tissue and other tissues (liver, lung, other organ tissue), form-fitting gels including polymerizable gels as otherwise described herein, cationic guar, and cationic starch as well as salts and esters thereof, or any combination thereof. A number of these gel materials also may be used for forming microcapsules pursuant to the present invention. In some embodiments, the biomaterial is polyethylene glycol diacrylate (PEG-DA) or other polymerizable gel, which may be further crosslinked after being administered to the patient or subject. It is noted that each of the biomaterials utilized in the present invention may be crosslinked to a greater or lesser extent or otherwise modified chemically to accommodate the engineered MSCs for encapsulation (e.g., microencapsulation by microspheres) or to provide support as a support matrix (scaffold). A preferred biomaterial for microencapsulation is polylethylene glycol diacrylate (PEG-DA) which is optionally and preferably crosslinked

The cells described herein can be grown in any suitable cell growth media. Cell growth media include, but is not limited to, mesenchymal stem cell culture media such as STEMPRO® MSC SFM (Life Technologies Corp.), ATCC® Mesenchymal Stem Cell solutions, TheraPEAK™ MSCGM-CD™ Medium (Lonza Corp.) or any number of other commercially-available or custom-formulated (e.g. serum-free) mesenchymal stem cell culture media, including Mesencult Basal Medium (Stem Cell Technologies, Inc.). Other cell growth media are well known in the art and can comprise a minimum essential medium plus effective amounts of optional agents such as growth factors, including fibroblast growth factor, such as basic fibroblast growth factor (bFGF), leukemia inhibition factor (LIF), glucose, non-essential amino acids, glutamine, insulin, transferrin, beta mercaptoethanol, and other agents well known in the art. Media includes, but is not limited to, commercially available media such as (Dulbecco's Modified Eagle Medium), or DMEM/F12 (1:1), among others, which may be supplemented with any one or more of L-glutamine, knockout serum replacement (KSR), fetal bovine serum (FBS), non-essential amino acids, leukemia inhibitory factor (LIF), beta-mercaptoethanol, basic fibroblast growth factor (bFGF), glial cell-line derived neurotrophic factor (GDNF) and an antibiotic, B27 medium supplement and/or N2 medium supplement. Cell media that can be used in the present compositions may also be found to be commercially available and can be supplemented with commercially available components, available from Invitrogen Corp. (GIBCO) and Biological Industries, Beth HaEmek, Israel, among numerous other commercial sources. Any one or more of these media may be combined with MSCs in compositions according to the present invention.

“One or more additional cell growth factors” or “growth factors” include, but are not limited to, hepatocyte growth factor (HGF), epidermal growth factor (EGF), β nerve growth factor (βNGF), retinoic acid, platelet-derived growth factor, transforming growth factor-β, insulin-like growth factor-1, vascular endothelial growth factor and fibroblast growth factor. One or more of these additional cell growth factors may be optionally included in compositions according to the present invention. The cell growth media can be modified as necessary to ensure the proper growth of cells.

Compositions and methods of treatment described herein may be used to treat or prevent one or more disorders selected from the group consisting of bone fracture, long bone nonunion, orthopedic soft tissue injury, spinal injury, skeletal and cartilage deficits, bone damage associated with primary bone cancers (e.g. osteocarcinoma), congenital bone malformation or nonunion, alveolar bone defects, cranial bone defects, facial bone defects, short bone defects, flat bone defects, irregular bone defects, sesamoid bone defects, cartilage defects, dentoalveolar defects, connective tissue defects and collagen membrane defects. In some embodiments, compositions and methods of treatment described herein may be used during guided bone regeneration, connective tissue grafts, or sinus lift procedures. In some embodiments, the compositions may be applied to a surgical implant or tissue graft that is implanted into the hard tissue or oral tissue of the subject.

In some embodiments, bone morphogenetic protein 2 (BMP2) can be used in compositions and methods described herein to achieve bone healing. Other proteins described herein, either alone or in combination with any other protein described herein, can also be used. Recombinant proteins can also be used. For example, recombinant BMP2 can induce rapid ossification in orthopedic applications. BMP-2 on its own can have a relatively short half-life and is administered at high dosages or continually maintained to promote extensive and expedited bone regeneration. Therefore, the compositions and methods described herein can be used to have a fast and maintained release/production of BMP-2 (or other proteins or factors described herein) as a therapeutic without the morbidity or side effects associated with bone grafting. The compositions and methods can also reduce recovery time and minimize future surgeries. In some embodiments, mesenchymal stem cells (MSCs) could be a vector for delivering BMP2, MSCs have several advantages: they can be easily harvested from adult bone marrow and adipose tissue, are immunomodulatory, have allogeneic tolerability, and are easily expanded in vitro and differentiate into bone even after long term culture.

In some embodiments, the cells expressing the bone regeneration proteins can be encapsulated. For example, the cell can be encapsulated in PEG or a derivative of PEG or other material. Cellular encapsulation with genetically engineered cells producing BMP2 in a PEG polymer for bone regeneration was developed to extend expression of BMP-2 in vivo. PEG is an attractive material for biomedical applications with biocompatibility in multiple tissues (Peppas 1981; Sawhney 1993; Bjugstad 2008; Wilson 2008; Liu 2010). Additionally, the mechanical properties of PEG can be altered to replicate that of soft tissue through the incorporation of extracellular matrix proteins and copolymers such as poly (propylene fumarate). As soft tissue injury often occurs at the same time as long bone injury healing involves the regeneration of both tissues. Mimicking in vivo soft tissue has been shown to be more permissive for physiological healing in creating an environment permissive for angiogenesis, a vital component for correct bone healing. PEG-DA can be biodegradable in tissues through manipulations of the peptide sequences linking PEG moieties which makes the structure cleavable through proteolytic processes allowing the polymer and encapsulated cells to be removed by the body during the healing process. Initial studies with BMP-2 transduced cells encapsulated in PEG have been shown to have advantages to unencapsulated cells through the extended presence at the site of treatment and increased induction of heterotopic ossification in the mouse. BMP-2 producing cells delivered through a scaffold or matrix also will have advantages over cells without the scaffold or matrix. Therefore, the compositions described herein can be used for applications in human or veterinary medicine for replacing or use in conjunction with current technologies for increasing the rate of bone healing.

As discussed herein, the cells can be cryopreserved. Cryopreserved cells can be stored and, therefore, in some embodiments be “ready to use” prior to the therapeutic application. Cryopreservation of a PEG-DA cell encapsulation preparation can also enhance and widen their therapeutic uses because the encapsulation and testing of preparations could be conducted well in advance in controlled good manufacturing practices (GMP) facilities for distribution to the clinical setting. Cryopreservation allows for thorough testing of the encapsulated MSCs with the ability to thaw samples for validating cell viability, therapeutic production, sterility, and microbead integrity to ensure that the highest quality production had been performed.

It has been found that cryopreservation did not adversely affect cell viability in both encapsulated MSCs and encapsulated genetically modified MSCs when compared to the freshly prepared samples. Additionally, there were no observable defects in the structure of the microbeads, which is in contrast to previous reports that used alginate microbeads. The cryopreserved encapsulated MSCs were capable of producing bone in the mouse model for heterotopic ossification as seen with the freshly prepared samples. These results indicate that cryopreservation is a valid method for preserving the viability and function of this valuable therapeutic.

The term “effective amount” is used throughout the specification to describe concentrations or amounts of compositions described herein or other components which are used in amounts, within the context of their use, to produce an intended effect. The formulations or component may be used to produce a favorable change in an injury or condition treated, whether that change is a remission, a favorable physiological result, a reversal or attenuation of a an injury or condition treated, the prevention or the reduction in the likelihood of an injury or condition occurring, depending upon the injury or condition treated. Where formulations are used in combination, each of the formulations is used in an effective amount, wherein an effective amount may include a synergistic amount. The amount of formulation used may vary according to the nature of the formulation, the age and weight of the patient and numerous other factors which may influence the bioavailability and pharmacokinetics of the formulation, the amount of formulation which is administered to a patient generally ranges from about 0.001 mg/kg to about 50 mg/kg or more, about 0.5 mg/kg to about 25 mg/kg, about 0.1 to about 15 mg/kg, about 1 mg to about 10 mg/kg per day and otherwise described herein. The person of ordinary skill may easily recognize variations in dosage schedules or amounts to be made during the course of therapy. That is, the effective amount can be determine, if necessary, by one of skill in the art. The amount of composition in the form of microspheres (the term microspheres in many instances may be used synonymously with the term microcapsules), a gel (including an in-situ gel or a polymerizable gel), a putty, or a cellular matrix was otherwise described herein which is used to treat a patient may range from about 50 to 100 μl (microliters) up to about 100 ml. or more when the composition is a gel or liquid (including a viscous liquid) and from less than 1 mg to upwards of several hundred grams or more (often about 10 mg to about 500 mg, often about 25 mg to about 100 mg), depending on the area to be treated and the severity of the condition, injury or disorder to be treated. The number of cells which have been engineered to express a bone regeneration protein included in compositions according to the present invention will range from as few as several thousand to upwards of hundreds of millions or more, again, depending on the severity of the condition, injury or injury to be treated. Micospheres are known in the art and are prepared using standard pharmaceutical methods and range in size from about 1-5 nanometers to hundreds of micrometers or more in size and may be delivered in that form directly or separately formulated in a gel, putty or cellular matrix for delivery to a site in the patient or subject to effect therapy.

The term “polymerizable gel” or “in-situ polymerizable gel” refers to a polymeric gel or a gel which comprises components which form gels in-situ because of their viscosity and/or reverse thermal gellation properties and which may be polymerized and/or crosslinked (by the addition of heat and/or light) after administration of the composition to the site of therapy in the patient or subject. Polymerizable gels include for example, polyacrylamides, polyacrylic acids, polyethylene glycol/acrylic copolymers, polyethylene glycol and/or polyester and/or polyester-co-polyethylene glycol copolymers which are end-capped with acrylic functional groups, among a larger number of polymers which may be employed. These polymers are well-known in the art. An “in-situ” gel is a gel which forms once administered to a patient, but is not necessarily polymerized, but is optionally polymerized after administration. These gels are widely varied and include polymers based upon pectin, xyloglucan, xanthum gum, chitosan, gellangum, alginic acid, carbopol, pluronics (e.g. F-127), other synthetic polymers including aliphatic polyesters, triblock polymer systems (polyester-block polyethylene glycol-polyester), polyacrylamides, and polyacrilic acids, among numerous others. A description of polymeric materials which are useful to provide scaffolds and micropheres pursuant to the present invention may be found in Nirmal, et al., International Journal of PharmacTech Research, Vol. 2, No. 2, pp. 1398-1408 (April-June, 2010) which is incorporated by reference herein.

The term “prophylactic” is used to describe the use of a composition described herein that reduces the likelihood of an occurrence of an injury or condition in a patient or subject. The term “reducing the likelihood” refers to the fact that in a given population of patients, the compositions and methods may be used to reduce the likelihood of an occurrence or recurrence of an injury or condition in one or more patients within that population of all patients, rather than prevent, in all patients, the occurrence or recurrence of an injury or condition. For example, the compositions described herein could be used prophylactically to reduce bone fractures.

The term “pharmaceutically acceptable” means that a composition described herein, or component thereof or a formulation comprised of such a composition, or an additive, diluent or excipient of a composition as described herein, is not unacceptably toxic to the subject to which it is administered.

Compositions described herein may also comprise a pharmaceutically acceptable diluent, carrier, solubilizer, emulsifier, preservative and/or adjuvant. Acceptable formulation materials are, for example, nontoxic to recipients at the dosages and concentrations employed. The pharmaceutical formulations may contain materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, polyethylene glycol (PEG), sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, e.g., sodium or potassium chloride, mannitol, or sorbitol); delivery vehicles; diluents; excipients and/or pharmaceutical adjuvants. See, for example, REMINGTON'S PHARMACEUTICAL SCIENCES, 18.sup.th Edition, (A. R. Gennaro, ed.), 1990, Mack Publishing.

Primary vehicles or carriers in a pharmaceutical formulation can include, but are not limited to, water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical formulations can comprise Tris buffer of about pH 7.0-8.5, or acetate buffer of about pH 4.0-5.5, which may further include sorbitol or a suitable substitute. Pharmaceutical formulations may be prepared for storage by mixing the selected composition having the desired degree of purity with optional formulation agents (REMINGTON'S PHARMACEUTICAL SCIENCES, Id.) in the form of a lyophilized cake or an aqueous solution. Further, the formulations may be formulated as a lyophilizate using appropriate excipients such as sucrose. Formulation components are present in concentrations that are acceptable to the site of administration. Buffers are advantageously used to maintain the composition at physiological pH or at a slightly lower pH, typically within a pH range of from about 5 to about 8.

In some embodiments, the therapeutic compositions may be in the form of a pyrogen-free, parenterally acceptable aqueous solution. Preparation involves the formulation of the desired microspheres, which may provide controlled or sustained release of the product which may then be delivered via a depot injection. Formulation with hyaluronic acid has the effect of promoting sustained duration in the circulation. In some embodiments, a composition comprising microspheres comprises microspheres that are at least 80, 85, 90, 91, 92, 93, 94, 95, 96, 97 98, or 99% the same size. In some embodiments, the microspheres are substantially the same size. The size can be determined by any method, and the uniformity of the population of the spheres can be determined once the size distribution of the spheres are determined. The spheres can also be sieved so that the composition contains a uniform size of spheres.

The pharmaceutical composition to be, used for in vivo administration typically is sterile. In some embodiments, this may be accomplished by filtration through sterile filtration membranes. In some embodiments, where the composition is lyophilized, sterilization using this method may be conducted either prior to or following lyophilization and reconstitution. In some embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

In some embodiments, once the formulation has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

In some embodiments, the compositions provided herein can be used for treating, preventing, or repairing bone or cartilage defects. Isolated compositions are also provided.

In some embodiments, the compositions comprise one or more biomaterials. In some embodiments, the one or biomaterials are selected from the group consisting of collagen, fibrin, silk, agarose, alginate, hyaluronan, chitosan, polyesters including polylactic-co-glycolic acid, polyethylene glycol, polyethersulfone, a peptide-based biomaterial, a ceramic-based biomaterial and mixtures thereof. In some embodiments, the compositions comprise one or more mesenchymal stem cells (MSCs), the MSCs comprising one or more expression vectors encoding one or more bone regeneration proteins. In some embodiments, the expression of the one or more bone regeneration proteins is operably linked to a promoter. In some embodiments, the promoter can be constitutive (e.g. always on). In some embodiments, the expression vector comprises a promoter that is tissue specific or cell specific or otherwise is an inducible promoter. For example, the promoter may only turn on the expression of the encoded protein in a specific cell or tissue type or be induced in the presence of certain compounds. In some embodiments, however, when the composition comprises polyethylene glycol the expression vector nucleotide sequence is not an adenovirus expression vector sequence or an adeno-associated virus expression vector sequence.

As discussed herein, in some embodiments, the composition comprises a nucleotide sequence encoding a bone regeneration protein or other protein of interest. In some embodiments, the composition comprises a nucleotide sequence that is complementary to an expression vector. In some embodiments, the composition comprises an expression vector nucleotide sequence. In some embodiments, the composition comprises a nucleotide sequence that is complementary to a viral expression vector or to a viral nucleotide sequence. In some embodiments, the sequence are integrated into a cell's genome. In some embodiments, the sequences are maintained extrachromosomally.

For example, if a lenti-based viral expression system is used, a part of the lentiviral RNA can be reverse transcribed and integrated into the cell's genome. This nucleotide sequence would be considered to be complementary to a viral expression vector or to a viral nucleotide sequence. This sequence would also be considered to be an expression vector nucleotide sequence. The nucleotide sequence encoding the bone regeneration protein or other protein of interest is not an expression vector nucleotide sequence. In some embodiments, the composition comprises a sequence that is from an AV or AAV expression vector or is complementary to AV or AAV. In some embodiments, the composition is free of a sequence that is from an AV or AAV expression vector or is complementary to AV or AAV expression vector. The sequences can be specifically complementary, which means that the sequences would hybridize with one another under high stringency conditions. High stringency conditions are well known in the art. In some embodiments, the sequences are at least 90, 91, 92, 93, 94, 95, 96, 97, 98, 99% identical to a sequence present in an expression vector (e.g. viral expression vector or virus).

As discussed herein, the bone regeneration protein can be a bone morphogenetic protein. In some embodiments, the bone regeneration protein is BMP-2, BMP-4, BMP-5, BMP-7, or any combination thereof. The bone regeneration protein can be a heterodimer or homodimer. In some embodiments, the heterodimer comprises BMP-2 and BMP-7. In some embodiments, the heterodimer comprises BMP2 and BMP4, BMP2 and BMP5, BMP4 and BMP5, BMP4 and BMP7, or BMP5 and BMP7. The other BMP proteins and nucleotide sequences encoding the same can also be used alone, as a homodimer, or part of a heterodimer. In some embodiments, the composition comprises 1, 2, 3, or 4 different bone morphogenetic proteins. The composition can also comprise other proteins or compounds that can be used to enhance the treatment of a bone or cartilage disorder. In some embodiments, the composition comprises SDF-1. In some embodiments, the SDF-1 is SDF-1α. In some embodiments, the composition comprises a nucleotide sequence encoding SDF-1α. In some embodiments, the composition comprises a single contiguous nucleotide sequence encoding both the bone regeneration protein(s) and SDF-1α.

In some embodiments, different expression vectors or different nucleotide sequences are used to express different proteins. That is, the nucleotide sequences encoding the different proteins are not linked through a covalent bond. In some embodiments, the different proteins are expressed from, or encoded by, a contiguous nucleotide sequence. In some embodiments, the expression of the different proteins are under the control of the same or different promoters. In some embodiments, the composition comprises an expression vector encoding IL-6, IL-8, and/or vascular endothelial growth factor; and/or an IL-6 polypeptide, an IL-8 polypeptide, and/or a vascular endothelial growth factor peptide. In some embodiments, the composition comprises prostoglandin E2. As discussed above, the composition can comprise a single expression vector or nucleotide sequence expressing each protein or multiple expression vectors or sequences that express one or more of the proteins. Other proteins can or factors can be used to enhance bone healing or cartilage healing.

In some embodiments, the composition is a microsphere or population of microspheres, gel, putty, or cellular matrix. In some embodiments, the MSCs are encapsulated by the one or more biomaterials. In some embodiments, the MSCs are encapsulated by PEG or derivative thereof. In some embodiments, the MSCs are not encapsulated by PEG or derivative thereof. In some embodiments, the composition is free of encapsulated MSCs.

In some embodiments, pharmaceutical compositions comprising any of the compositions described herein are provided. In some embodiments, the pharmaceutical compositions comprise a pharmaceutically acceptable excipient. In some embodiments, the composition is suitable for injection. In some embodiments, the composition is a putty, gel, or solid. The form of the composition can be modified depending upon the use and method of administration. In some embodiments, the composition is formulated for transdermal delivery, and would be referred to as a transdermal composition. In some embodiments, the transdermal composition comprises a patch for transdermal delivery.

In some embodiments, methods of treating or preventing a bone or cartilage disorder are provided. In some embodiments, the method comprises administering to a subject having the bone or cartilage disorder any composition described herein. In some embodiments, the method comprises contacting the location of the bone or cartilage defect. In some embodiments, the method comprises thawing the composition prior to administering. As discussed herein, the bone or cartilage disorder can be a bone fracture, long bone nonunion, orthopedic soft tissue injury, spinal injury, skeletal and cartilage deficits, bone damage associated with primary bone cancers including osteocarcinoma, congenital bone malformation or nonunion, alveolar bone defects, cranial bone defects, facial bone defects, short bone defects, flat bone defects, irregular bone defects, sesamoid bone defects, cartilage defects, dentoalveolar defects, connective tissue defects, or collagen membrane defects.

In some embodiments, the method comprises administering to the subject concomitantly or sequentially one or more synthetic bone proteins and/or performing a bone graft on the subject.

In some embodiments, the subject has adenovirus immunity prior to administering the composition. In such a situation, the subject can be treated with a composition that does not comprise an adenovirus expression vector or a MSC that was transfected or infected with an adenovirus or adenovirus expression vector. A subject that has “adenovirus immunity” can be a subject that has antibodies against adenovirus. In some embodiments, a subject that adenovirus immunity has neutralizing antibodies against adenovirus.

In some embodiments, the subject has suffered a bone fracture and is injected at the fracture site with a pharmaceutically effective amount of any composition described herein. In some embodiments, there is little or no soft tissue including tendon, ligament, fat, and or muscle that surrounds the fracture. These and other aspects of the embodiments described herein described further in the following non-limiting examples. Each of the references, patents, and accession numbers provided herein are incorporated by reference in their entirety.

### EXAMPLES

### Example 1

### Preparation of Non-Rodent Cells for Overexpression of BMP and Cryopreservation

### Summary

In the experiment of this example, mesenchymal stem cells (MSCs) were used as a delivery system for transgene production. MSCs were chosen due to their ease of harvest, replication potential and immunomodulatory capabilities. MSCs used were from sheep and pig due to their use as large animal models for bone nonunion. The cells were also microencapsulated. The cryopreservation of these microencapsulated therapeutic MSCs did not affect their cell viability, transgene BMP-2 production or ability to initiate bone formation in a mouse model for heterotopic ossification when compared to freshly prepared samples. Additionally, the microspheres showed no appreciable damage from cryopreservation when examined with light and electron microscopy. While the viability of MSCs in all conditions was reduced by 4 days following encapsulation when compared to the day of encapsulation, genetically modified encapsulated MSCs at 4 days post-encapsulation also showed additional reduction in viability. These results validate the use of cryopreservation in preserving the viability and functionality of PEG encapsulated BMP-2 transduced MSCs.

### Material and Methods:

Pig and sheep are a suitable models for human bone studies with long bone dimensions (Raab 1991; Newman 1995) and structure (Mosekilde 1987; Thorwarth 2005) that are similar to man. Pig has been shown to have similarities in bone remodeling (Thorwarth 2005) while sheep provide a comparable model for bone in growth into osteoconductive biomaterials (Willie 2004). Using MSCs isolated from both pig and sheep cryopreserved PEG encapsulated MSCs expressing BMP-2 were examined. The cryopreservation of the cells within the polymers showed no reduction in viability in comparison to non-preserved encapsulated MSCs, and the encapsulated cellular spheres showed no physical damage resulting from cryopreservation. It was also found that cell lines from various donors may have different potentials in genetic modification and transgene production. Using this process genetically modified cryopreserved MSCs producing BMP-2 maintained function as seen through initiation of bone formation in an in vivo model for heterotopic ossification. These results demonstrate PEG microspheres can be used for “ ” therapeutic use.

### MSC Isolation and Culture:

Porcine MSCs were isolated previously (Bosch 2006) and ovine MSCs were isolated with the same plate adherency techniques from healthy female ewes as previously described. Briefly, MSCs were isolated from bone marrow aspirates with 0.25 mls ACD per ml of bone marrow. MSCs were plated by mixing in a 3/5 ratio with MSC culture medium:Alpha-Minimun Essential Medium (Gibco), 10% defined fetal bovine serum (Hyclone), 2 mM L-glutamine, 50 U/ml penicillin, 50 μg/ml streptomycin (all from Gibco/Invitrogen) and plating on tissue culture flasks. Cultures were maintained at 37° C. and at 5% CO2. MSCs were harvested using 0.05% trypsin (Gibco) and replated at 5,000 cells/cm² upon reaching 80-90% confluency (60,000-75,000 cells/cm²).

### Lineage Differentiation:

Differentiation was performed using previously established protocols (Bosch 2006) with the following alterations: for adipogenic and osteogenic differentiation, 36,000 cells/cm² were plated in 6 well plates. MSCs were allowed to reach confluency and then switched to adipogenic or osteogenic medium: adipogenic differentiation was initiated in induction medium:Dulbecco's Modified Eagle Medium (DMEM) high glucose (Invitrogen), Pen/Strep (Gibco), 1 μM dexamethasone, 10 μg/mL insulin, 200 μM indomethacin, 500 μM 3-isobutyl-1-methyl-xanthine (Sigma), and 10% FBS (Hyclone) for 3 days followed by 14 days in differentiation medium: DMEM high glucose, Pen/Strep, 10 μg/mL insulin, and 10% FBS. Differentitiated plates were stained with 0.7% Oil Red 0. Osteogenic differentiation was performed using HyClone Advance STEM Osteogenic Differentiation kit (Thermo Scientific) with medium changes every third day for 21 days and samples were stained with Von Kossa. For chondrogenic differentiation potential of MSCs, 3×10⁶ cells were pelleted in 15 ml conical tubes and then changed to chondrogenic medium DMEM (high glucose), 100 nM dexamethasone, Pen/Strep, 50 μg/mL ascorbic acid, 40 μg/mL L-proline, 1×ITS+1 supplement, 1 mM sodium pyruvate (Sigma), and 10 ng/mL TGF-β3 (R&D Systems). Medium was changed every third day for 14 days. Micromasses were stained with Alcian Blue.

### Proliferation:

Proliferation was determined using manual cell counts with 0.4% trypan blue (Sigma) live/dead exclusion staining, and only live MSCs were counted. MSCs were plated at 6,000 cells/cm² and harvested for counts 12 hours following plating. This initial count was deemed time 0 and MSCs were harvested and counted at 12, 24, 36, 48 and 60 hours after the initial count. Counts were performed in triplicates.

### Microencapsulation:

Using techniques previously described (Olabisi 2010), MSCs were harvested using 0.05% trypsin and counted on a hemocytometer using 0.04% Trypan Blue (Sigma) staining for live/dead exclusion. 3.5×10⁴ MSCs/ul were suspended in aqueous hydrogel solution containing 0.1 g/mL 10 kDa PEG-DA, 1.5% (v/v) triethanolamine/HEPES buffered saline, 37 mM 1-vinyl-2-pyrrolidinone, 0.1 mM eosin y, 9 mM pluronic acid. For photo initiation, 1.17M 2,2-dimethoxy-2-phenyl acetophenone was dissolved in 1-vinyl-2-pyrrolidinone and 3 uL of this solution was added per mL of sterile mineral oil (Sigma-Aldrich). Hydrogel/cell suspension was mixed with mineral oil containing the photoinitiator and vortexed for 2 seconds while being exposed to white light followed by another 18 second exposure with mild mixing. Microencapsulated MSCs were separated from the oil with four washes in MSC culture medium with 5 minute centrifugation at 1350 RPM and decanting between washes.

### Viability Assays:

Cell viability was assessed using the LIVE/DEAD Viability/Cytotoxicity Kit for Mammalian MSCs (Invitrogen). Images were taken using TCS SP5 Spectral Confocal Microscope (Leica). 3 sets of images were taken per condition with 30 images in each set with an average of 87,210 cells being counted in each treatment using Image J (NIH).

### Cryopreservation and Thawing:

MSCs and microspheres were frozen in MSC culture medium containing 10% DMSO. The MSCs were frozen in controlled rate freezing containers, Mr. Frostys (Nalgene labware) for 4-24 hours at −80° C. and then transferred to liquid nitrogen. Vials were thawed in a 37° C. waterbath with constant swirling. The MSCs were resuspended with medium immediately following loss of ice from cell/microbead suspension. To limit confounding factors microbeads were thawed using a ratio of twenty percent physical cell loss. This number was established on the percentage of cells lost during cryopreservation and thawing processes.

### Adenoviral Transduction Optimization and BMP-2 Quantification:

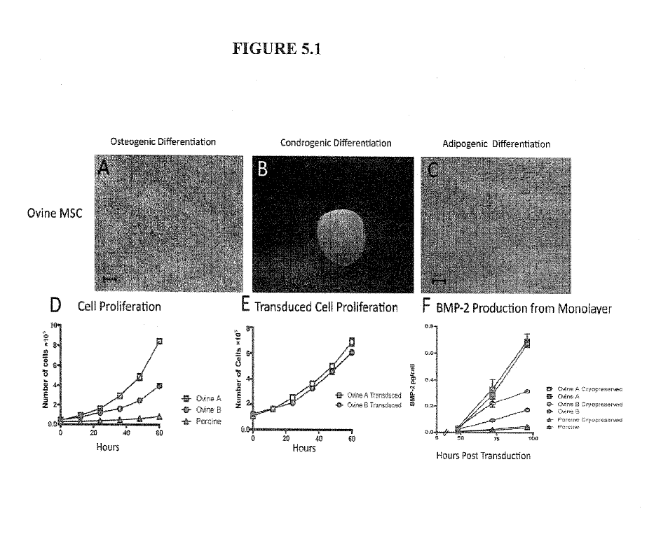

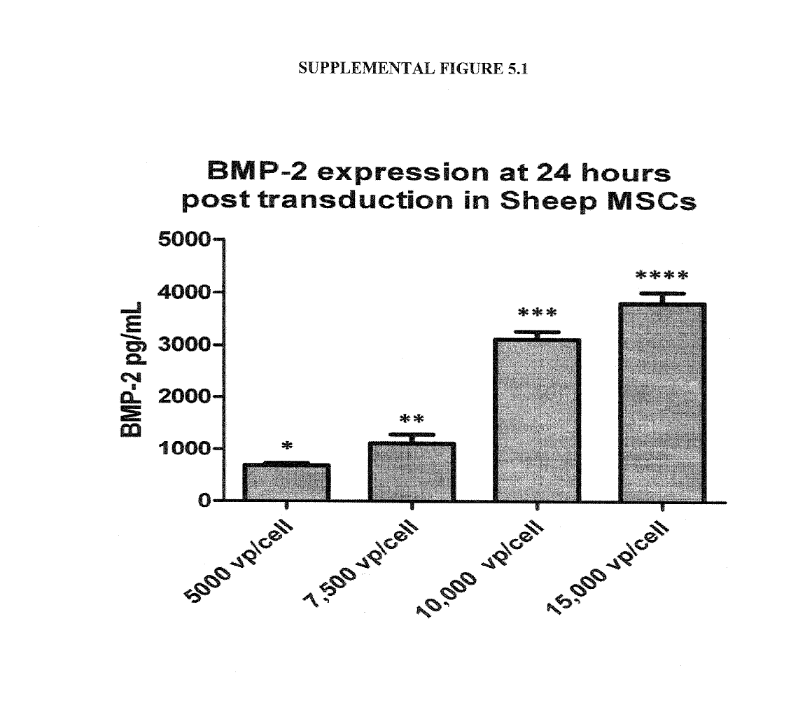

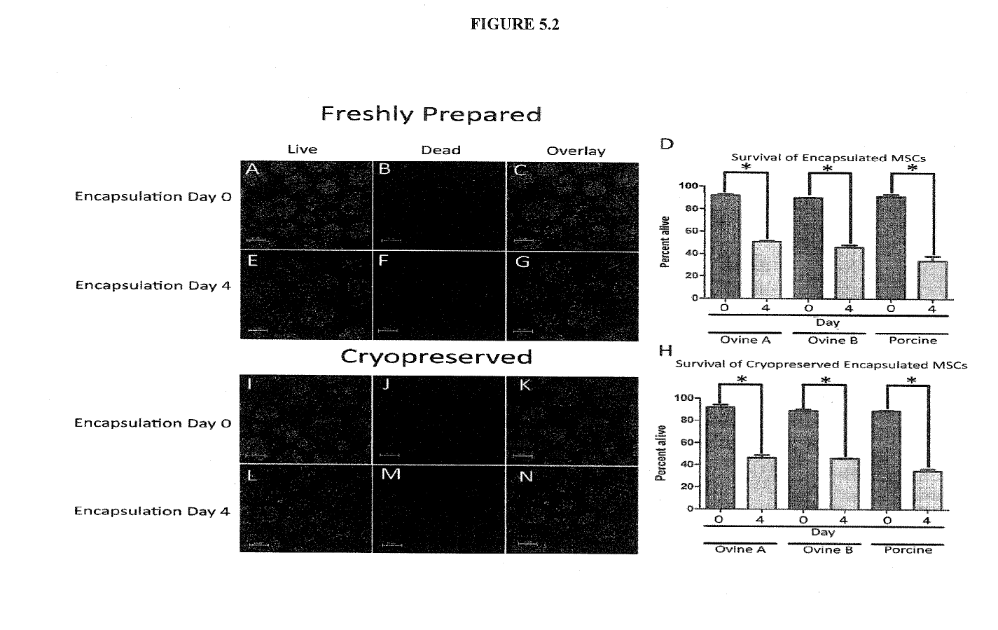

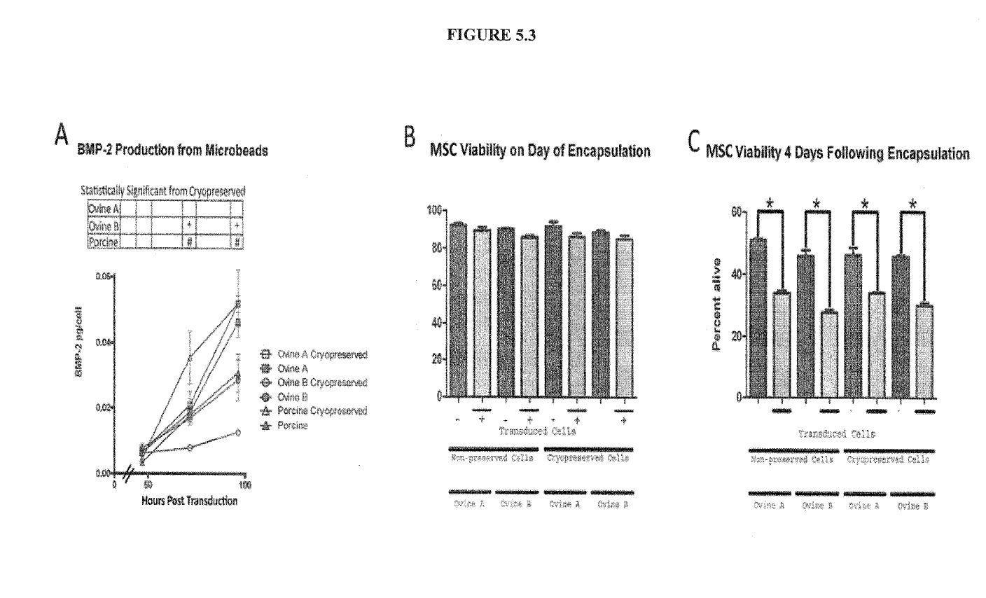

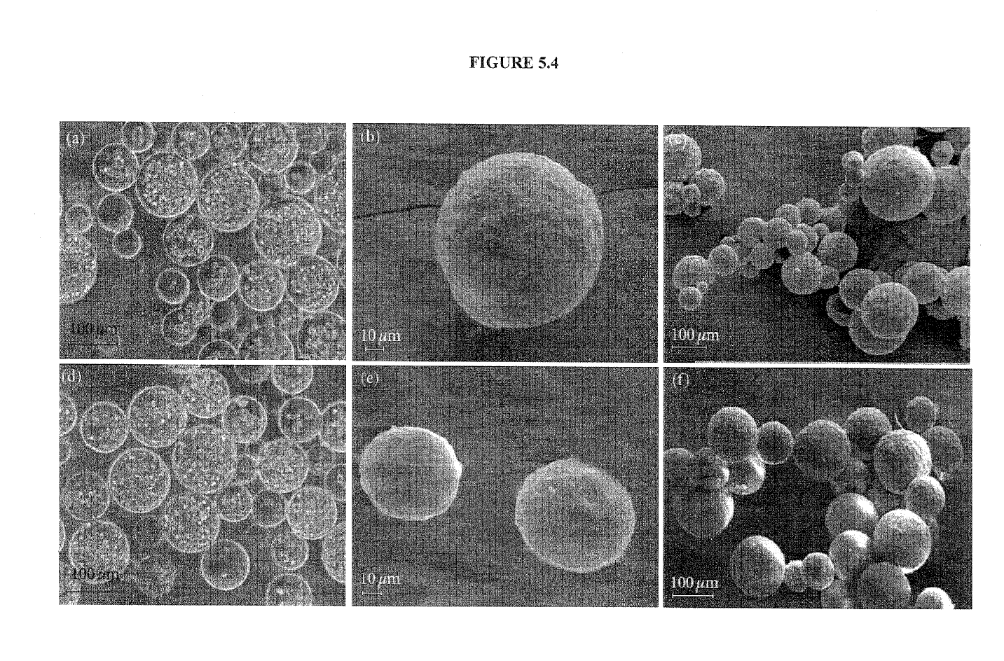

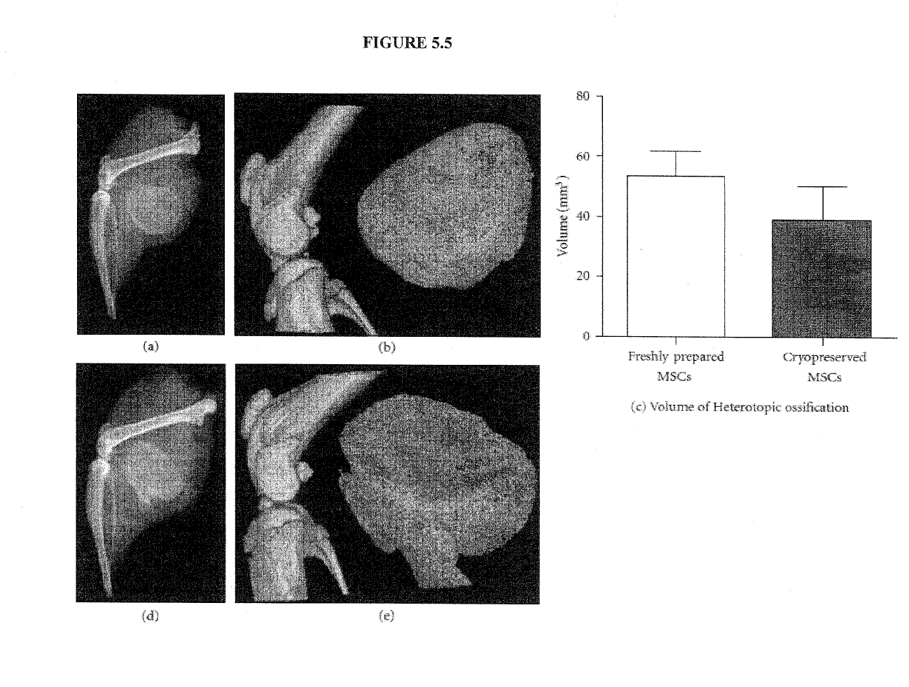

First generation human type 5 adenoviruses containing the E1-E3 deletion were constructed with human cDNA for BMP-2 inserted in the E1 region. See, Olmsted, E. A. et al., J Cell Biochem 82 (1), 11 (2001). MSCs were harvested and plated one day prior to transductions. Transductions were preformed as described previously (Bosch 2006) with minor changes. Upon reaching a density of 36,000 cells/cm² the MSCs were prepared for transduction. To increase cell-viral interactions transductions were performed in reduced medium volumes. Medium was changed with replacement of 32% of normal culture volume of MSC culture medium. Transduction medium was made equaling 20% of normal culture volume with Alpha MEM medium with 2 mM L-glutamine and mixed with 0.72% Genejammer (Agilent Technologies) and allowed to incubate for 5 minutes at room temperature. The virus was then added to the transduction medium and allowed to incubate for 10 minutes at room temperature. For optimization of BMP-2 transduction, transductions were performed using 5,000, 7,500, 10,000 and 15,000 vp/cell (Supplementary FIG. 5.1). The remainder of the experiments were performed with 15,000 vp/cell. The transduction mixture was then added to the cell culture drop wise around the plate. After four hours the culture volume was brought up to normal volume with MSC culture medium. MSCs were harvested 24 hours after the transduction. The MSCs were harvested and replated at 36,000 cells/cm² or encapsulated then replated at 36,000 cells/cm². BMP-2 was quantified from harvested medium using a BMP-2 elisa (R&D systems).

### Scanning Electron Microscopy and Light Microscopy:

Both freshly prepared and cryopreserved microbeads containing ovine MSCs were immersion fixed using 2.0% gluteraldehyde in PBS for one hour. The MSCs were washed three times with PBS and postfixed in 1% osmium tetroxide diluted in 5% sucrose and PBS for 45 minutes. The microbeads were washed three times with distilled water and then carried though an alcohol dehydration series. The MSCs were critically point dried using a Samdri model 780-A (Tousimis). A 153 Å thick coating of gold was placed on the samples using SPI Module Sputter Coater (Structure Probe). The images were taken on 1450EP environmental Scanning Electron Microscope (Carl Zeiss).

### Heterotopic Bone Assay:

Female non-obese diabetic/severely compromised immunodeficient mice (NOD/SCID; 8-12 weeks old; Charles River Laboratories) were injected with 3×10⁶ microencapsulated MSCs either freshly prepared or cryopreserved and thawed from ovine A MSCs and ovine B MSCs. Microbeads were injected into the hind limb quadriceps of 3 mice per group (n=12). Animals were euthanized at 2 weeks and x-rayed. The tissue was then harvested and fixed in formalin.

### Graphical Representation and Statistics:

Graphs were made in Prism (Graphpad) and all statistics were also done in Prism. Statistics comparing BMP-2 production were performed using 2-way ANOVA with Bonferonni post test. Viability comparisons were done with 1-way ANOVA using Tukeys post test. Doubling times were calculated using the exponential growth equation in Prism and comparison of doubling times were done with 1-way ANOVA with Bonferroni post test.

### Results:

Isolated Ovine MSCs are Capable of Adipogenic, Chondrogenic and Osteogenic Differentiation: Lineage differentiation of Porcine MSCs used in this study were previously validated (Bosch 2006). To determine the potential of ovine MSCs to produces MSCs of the adipogenic, chondrogenic and osteogenic lineages MSCs were differentiated. Ovine MSCs isolated through plate adherence from bone marrow aspirates were capable of adipogenic, chondrogenic and osteogenic differentiation (FIG. 5.1A, 5.1B, 5.1C). Ovine MSCs underwent 21 days of osteogenic differentiation and showed evidence of calcium deposition as seen through Von Kossa silver nitrate staining (FIG. 5.1A). After 14 days of condrogenic differentiation the micromasses exhibited sulfate proteoglycans as seen through Alcian Blue staining (FIG. 5.1B), indicating the presence of condrocytes. At 17 days of adipogenic differentiation, lipid droplets were visible within MSCs through Oil Red 0 staining (FIG. 5.1C), validating the capacity of these derived MSCs to differentiate into all three mesenchymal stem cell lineages.

Cell lines exhibited proliferation differences with and without adenoviral transduction: During the expansion phase it was noted that the ovine A MSCs reached confluency faster than the ovine B MSCs and porcine MSCs. Using 5 counts at 12 hour intervals proliferation rates were determined. Ovine A MSCs had a doubling time of 15.19 (+/−0.705) hours (R²=0.9813), Ovine B MSCs had a doubling time of 19.65 (+/−1.545) hours (R²=0.9867) and porcine MSCs had a doubling time of 34.54 (+/−3.175) hours (R²=0.9755). The doubling times from each line were all statistically different (p<0.05) (FIG. 5.1D). To understand the effect transduction had on the proliferation rates, doubling times of ovine A and ovine B MSCs were determined by plating the MSCs 24 hours after transduction and counting as described for the nontransduced cells. Adenoviral BMP-2 transduced ovine A and ovine B MSCs showed a significant reduction in the proliferation rates from the non transduced MSCs (p<0.05) (FIG. 5.1E) with a doubling time of 24.07 (+/−2.065) hours (R²=0.9827) and 25.37 (+/−2.16) hours (R²=0.9820) respectively.

BMP-2 Adenoviral Transduction of MSCs Had a Significant Donor Effect: To determine the ability of the MSC to produce BMP-2 following adenoviral transduction and the effect of cryopreservation on BMP-2 production, monolayers of MSCs were transduced with 15,000 viral particles/cell. 15,000 vp/cell was chosen based on the highest BMP-2 production from optimization of 5,000, 7,500, 10,000 and 15,000 vp/cell (p<0.05) (supplementary FIG. 5.1). The MSCs were replated 24 hours after transduction or cryopreserved. Medium was harvested from cultures every 24 hours for 72 hours and quantified for BMP-2 expression (FIG. 5.1F). The lines showed a significant donor effect (P<0.001) with ovine A MSCs producing the most BMP-2. Ovine B MSCs had a significant increase in BMP-2 expression from cryopreserved samples at 48 and 72 hours (P<0.001).

Cryopreserved Encapsulated MSCs Demonstrate High Levels of Cell Viability and Sustain BMP-2 Production: To examine the effect of cryopreservation on the survival of encapsulated MSCs, the viability of MSCs encapsulated in PEG-DA were assessed using a live/dead assay which stains the cytoplasm of live MSCs with Calcein AM (FIG. 5.2A, 5.2E, 5.2I, 5.2L) and the dead MSCs DNA with Ethidium Homodimer (FIG. 5.2B, 5.2F, 5.2J, 5.2M). No statistical difference was seen in the cell viability between the freshly prepared MSCs and the cryopreserved MSCs, but a significant reduction in cell viability was observed between day 0 and day 4 post-encapsulation in both freshly prepared and cryopreserved microspheres (p<0.0001) (FIG. 5.2D, 5.2H). When encapsulated, BMP-2 transduced MSCs produced a reduced quantity of BMP-2 (FIG. 5.3A) when compared to monolayer BMP-2 transduced MSCs (FIG. 5.1B) at 72 and 96 hours post-transduction (p<0.05). Porcine encapsulated BMP-2 producing MSCs showed an increase in BMP-2 production at 72 (p<0.01) and 96 hours (p<0.001) post transduction, and cryopreserved ovine B MSCs had a reduction in the quantity of BMP-2 produced at 72 and 96 hours post transduction (p<0.01) (FIG. 5.3A). Ovine A MSCs had no difference between the cryopreserved and freshly prepared encapsulated MSC BMP-2; Within the ovine lines, ovine A MSCs produced significantly more BMP-2 than ovine B MSCs at 96 hours post transduction (P<0.01) under both conditions (FIG. 5.1B, 5.3A). BMP-2 transduction has no effect on viability immediately following encapsulation (FIG. 5.3B), but the BMP-2 encapsulated MSCs did have reduced viability at day 4 (p<0.05) when compared to the non-modified MSCs (FIG. 5.3C).

Microspheres Do Not Show Surface Damage Resulting from Cryopreservation: The integrity of the microspheres was examined following cryopreservation though scanning electron microscopy and light microscopy. The light microscopy images (FIG. 5.4A, 5.4D) show the perimeter of the bead containing encapsulated ovine MSCs as being one contiguous surface with no rough edges. Additionally high magnification images of the encapsulated ovine microbeads demonstrated that the spheres possess contiguous surface with no loss of integrity (FIGS. 5.4B, 5.4E). Cryopreservation did not result in any changes in MSC encapsulated surface morphology (FIG. 5.4C, 5.4F).

BMP-2 Transduced MSCs Produce Bone in Murine Models Following Cryopreservation: In an animal model for heterotopic ossification, BMP-2 transduced encapsulated microbeads produce similar quantities of bone. 2 weeks following injection into NOD/SCID, when viewed by X-ray analysis, both with and without cryopreservation (FIG. 5.5).

### Discussion:

A major hurdle in making clinical treatments for these diseases is finding a way to make the therapeutics widely applicable and readily available for medicinal uses. The recipient of a cell therapy, like organ transplantation, is at risk for graft rejection and cell encapsulation is widely used to attempt to modulate this immunological process. In this study it was demonstrated for the first time that primary MSCs could successfully be cryopreserved in PEG microbeads. This is a surprising and unexpected finding and demonstrates that the composition can be used as a therapeutic.

The combined PEG-DA microbead encapsulation and cryopreservation method that yields high MSC viability post-thaw, similar to alginate and sodium cellulose sulfate cell encapsulation techniques (Dixit 1993; Li 2002; Stiegler 2006; Mayer 2010). However, unlike previous reports of damage in alginate capsules during the cryopreservation process (Chin Heng 2004; Heng 2004; Van Lieshout 2011), PEG micropheres did not show any appreciable damage upon removal from cryopreservation when examined by both light and scanning electron micropscopy. Compromises in the integrity of the microbead can result in exposure of the encapsulated MSCs and initiation of an immune rejection (Rabanel 2009). Cryopreserved encapsulated BMP2 transduced MSCs maintained their potential to form bone in a mouse model for heterotopic ossification, indicating that these preparations can be stored with no adverse effects on quality of the treatment. These composition can be used for production of a human based product at, for example, GMP facilities with distribution to clinics. The viability of the primary MSCs was adversely affected by adeno genetic modification and stressors in transduction and encapsulation process.

As discussed herein, the viability of the cells can be increased with the inclusion of extracellular matrix proteins or using a different cell line with improved viability.

The MSC line used for adenoviral BMP-2 transduction can impact the amount of BMP-2 expressed. There was donor variation which affected both the rates of proliferation and BMP-2 production from the MSC lines, but different cell lines can be chosen for different applications that require differing amounts of BMP-2 production. For example, the amount of BMP-2 expression and rate of proliferation followed the same trend, shorter cell cycle time can be used an indicator of cell lines which are more amenable to higher rates of transduction, and, therefore expression of BMP-2 or other bone morphogenetic proteins. As adenovirus is effective at transducing cells in the S phase, cells with a shorter doubling time would be more likely to pass through S phase in the presence of active virus. There was less difference in BMP-2 production between all lines following encapsulation, but a difference between the ovine lines was still observed. This again indicates that MSC line to line variability significantly impacts the amount of BMP-2 produced, and that BMP2 expression optimization may need to be conducted for each batch or lot of MSC collected regardless of prior experience. Cryopreservation will potentially facilitate the storage of large lots of characterized product.

### Conclusions:

Microencapsulation of MSCs holds much promise for therapeutics in diseases without current effective treatments. To move these treatments forward, methods for preserving and long term storage of encapsulated MSCs to allow for “off the shelf” therapeutics is necessary. The cryopreservation of PEG encapsulated MSCs did not reduce cell viability between the cryopreserved and freshly prepared MSCs both with and without genetic modification and did not demonstrate any physical damage resulting from the cryopreservation process. Cryopreservation does not induce any negative effects on the encapsulated MSCs both with and without transduction and has no effect on the ability of the transduced cells to form bone; however, the encapsulated MSCs did have reduced viability following adenoviral transduction indicating a need for incorporating methods that increase viability of encapsulated MSCs to prolong protein production. Donor to donor variability results in significant transgene production making cell line choice important for optimizing gene expression. This demonstrates that PEG encapsulated MSCs have potential for being used as a treatment method for clinical applications. See Mumaw, et al., “Rapid Heterotrophic Ossification with Cryopreserved Poly(ethylene glycol-) Microencapsulated BMP2-Expressing MSCs”, Int J Biomater. 2012; 2012:861794. Epub 2012 Feb. 7 (PMID:22500171), the complete contents of which are hereby incorporated by reference.

### Example 2

### Suppressing the Adenoviral Introduced Gene Expression in MSC Immune Response Generates Improved Bone Formation/Use of “Fracture Putty” to Repair Long Bone Fractures in Sheep

The objective of this experiment was to determine if a construct consisting of BMP-2 transduced mesenchymal stem cells contained in a PEG-DA polymer (BMP-2-MSCs) could enhance bone formation in specific bone defects created in sheep.

To test this construct we created bone defects that ranged from minor bone trauma to complete critical sized defects with the ultimate goal being that of repairing a critical size defect in a long bone such as the tibia or femur. To test the osteogenic ability of BMP-2-MSCs we utilized 2 different types of bone injuries and observed healing over a 28-30 day period. The results obtained using a total of 10 sheep are described herein.

All of the sheep were purchased from the University of Georgia Department of Animal and Dairy Science and delivered to Building 11 at the College of Veterinary Medicine in groups of 4 animals at a time. Sheep were housed in groups of 2-4 animals/stall and day to day husbandry was provided by Animal Resources. All animals were acclimated for 7 days prior to performing any procedure and were given physical examinations daily by project staff.

### Defect One: Tibial Unicortical Defect.

For the first phase of the study 6 sheep received injections of BMP-2-MSCs near a proximal tibial uni-cortical defect. To create this defect sheep were anesthetized and placed in dorsal recumbency. The proximal aspect of the tibia was clipped and surgically prepared. Using a percutaneous approach the proximal tibia was drilled with a 4.5 mm drill bit aimed at the proximo-dorso-lateral aspect of the bone. The drill was advanced into the bone to penetrate the cortex and into the trabecular metaphyseal bone. 3 to 4 mls of hydrogel containing BMP-2-MSCs were then delivered to the area of the defect and within the drill hole. The skin was closed with skin staples.

A similar defect was made in the contralateral limb. This defect was not treated with BMP-2-MSCs and served as a control. Following recovery from anesthesia all sheep tibias were radiographed at 14 and 28 days post-operatively to evaluate bone formation in treated vs. control limbs.

### Results:

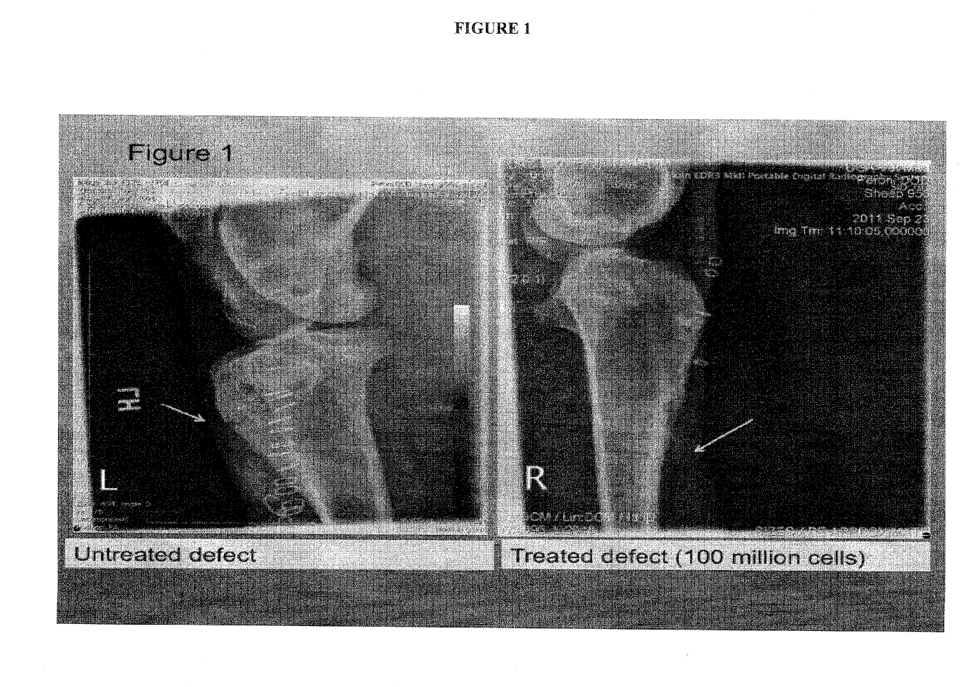

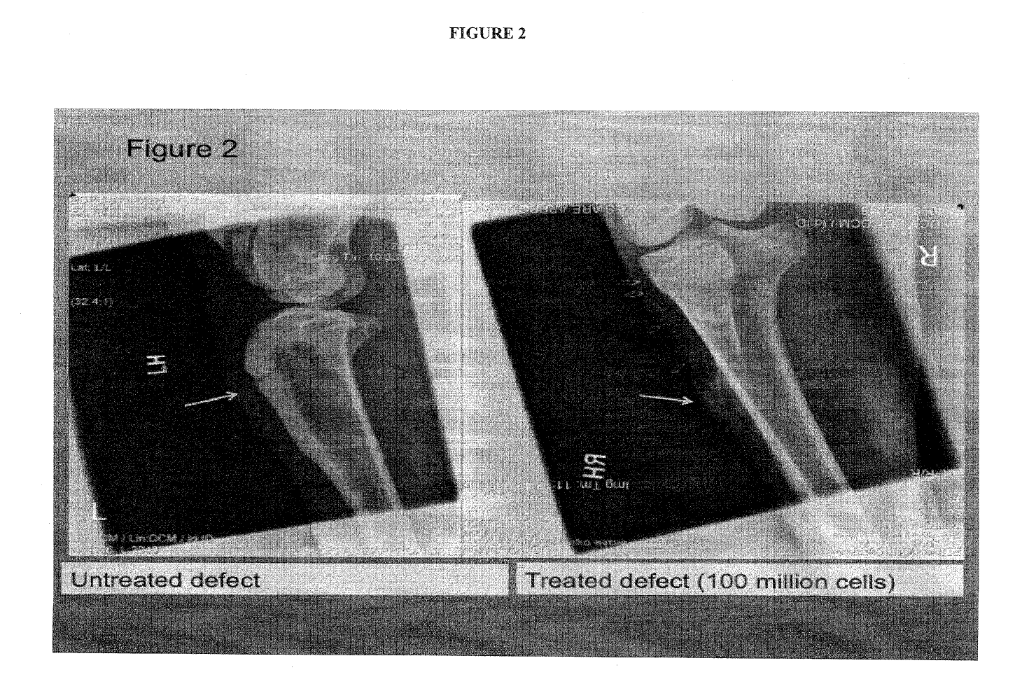

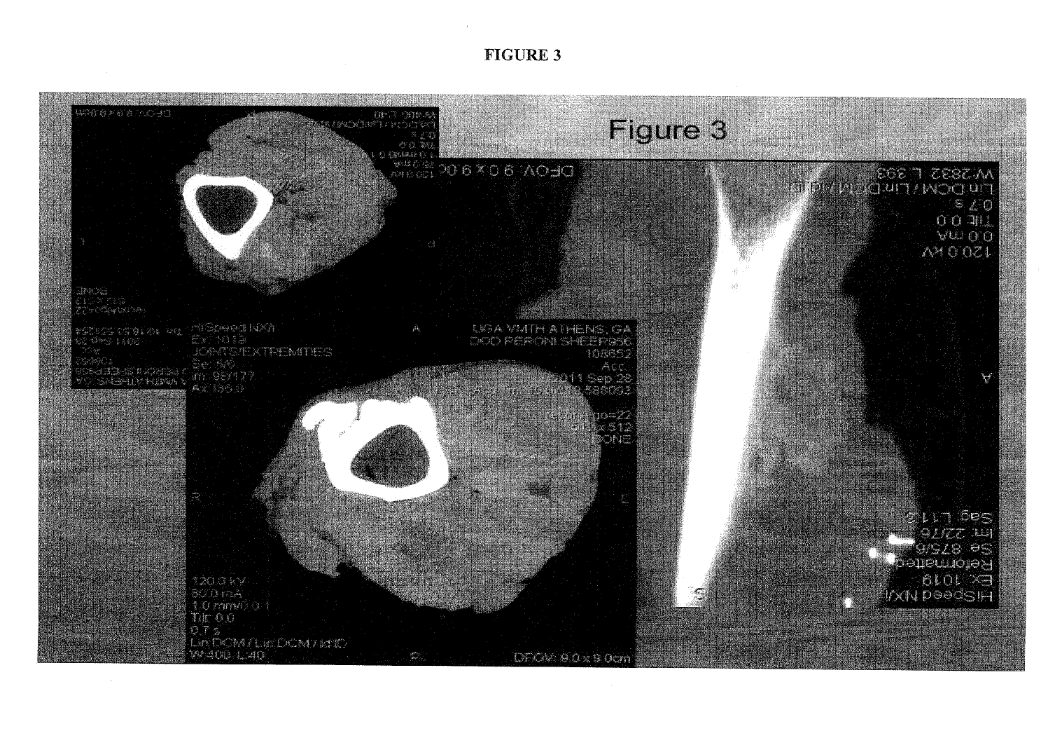

Bone formation was observed in the treated limbs of 3 out of 5 animals by 28 days. Bone was seen within the cortical defect and protruding beyond the surface of the bone in an orientation consistent with the downward flow of the injected hydrogel. See FIGS. 1-3.

### Defect Two: Ulnar Osteotomy.

After obtaining successful bone formation using the above reported bone defect, we selected an osteotomy model that would allow to test the fracture healing potential of our contract. This was preformed using 5 more sheep.

Cyclosporine (5 mg/kg) orally was administered prior to surgery to 4 of the 5 sheep and was continued every 24 hours for a total of 5 days post-operatively After clipping of the hair and surgical preparation of an area extending from the carpus proximally to above the shoulder area, the ulna was approached with a longitudinal incision located along the caudal aspect of the bone involving skin and underlying aponeurosis. The deep flexor muscle bodies were located and the ulnar and deep heads of this muscle were divided and separated using a Gelpi forceps. The bone was identified and a 4 hole 3.5 mm dynamic compression plate (DCP) was positioned along its shaft and measured. The proximal 2 screws (3.5 mm cortical screws) were placed thorough the plate into the bone in a neutral position using appropriate orthopedic instrumentation and were left loose. The plate was rotated out of the way of the bone and a 1 cm bone defect was created with a bone saw in an area of the ulna that would lie immediately beneath the mid-portion of the plate. Bone debris was cleared. The plate was rotate back onto the ulna and the two distal screws were placed in similar fashion as the proximal screws. All implants were tightened. Muscle was then closed with 2-0 PDS in a simple continuous pattern to cover the plate. Fascia was closed similarly. Skin was apposed with skin staples. The incision was covered with a non-adherent sterile bandage and elastikon bandage was applied for recovery.

A unicortical defect of the tibia was also made in these sheep as described previously. Once the sheep recovered from anesthesia they remained in Animal Resources housing for 48 hours at which point they were sedated and the previously created ulnar osteotomies treated with BMP-2-MSCs Cell treatments and controls were done as outlined in the table below. Control means no treatment and graft means cancellous bone graft (gold standard for bone healing).

| Sheep ID | Cyclosporine | LF (ulna) | RH (tibia) |
|---|---|---|---|
| 6184 | Yes | BMP-2-MSCs | control |
| 6240 | Yes | control | BMP-2-MSCs |
| 6241 | Yes | BMP-2-MSCs | graft |
| 5784 | Yes | graft | BMP-2-MSCs |
| 6110 | No | BMP-2-MSCs | BMP-2-MSCs |

### Results.

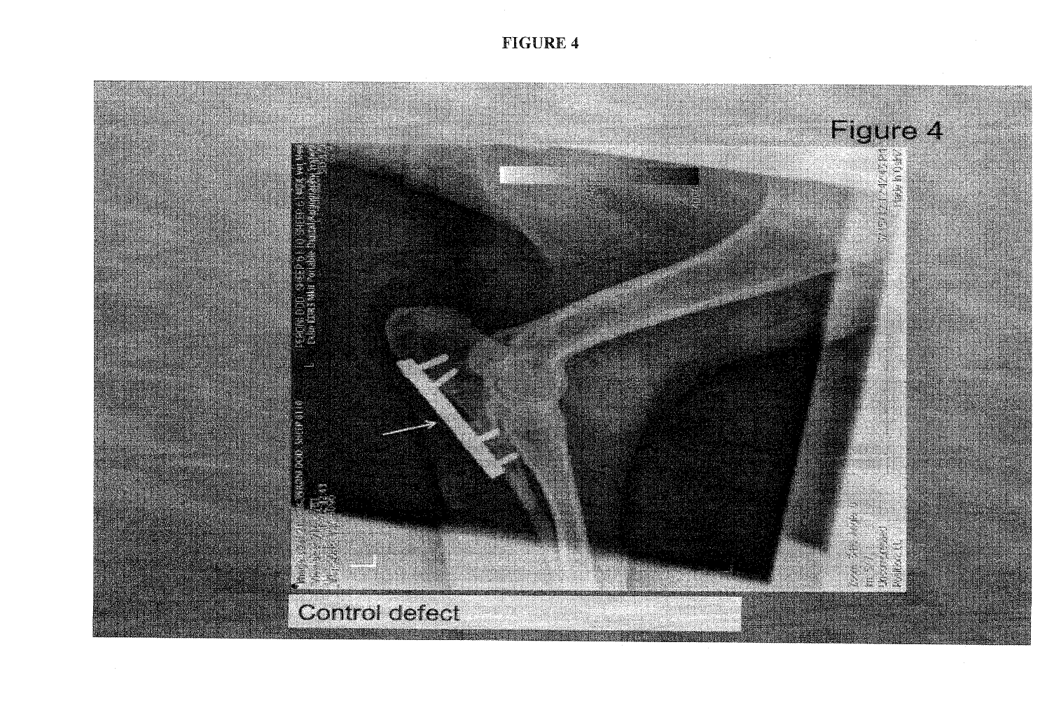

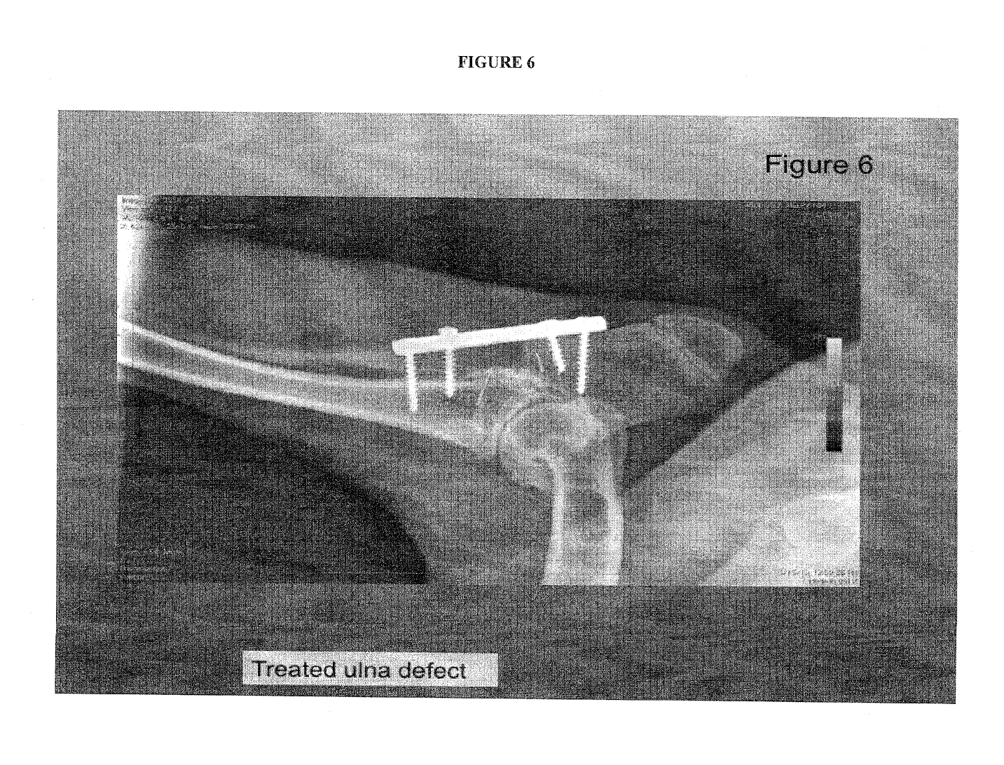

Radiographs were taken 26 days from injection in all sheep. No bone formation was observed in the non-cyclosporine animal (6110) despite treatment of ulna and tibia with BMP-2-MSCs. FIG. 4. The ulna defect that received a cancellous graft (5784) filled with new bone. In the same animal (5784), the tibia treated with BMP-2-MSCs showed intense pericortical bone formation. FIG. 5. One sheep (6184) out of 4 treated with cyclosporine showed bone deposition within the ulna defect following injection with BMP-2-MSCs. This was inferior to that observed with cancellous bone grafting. FIG. 6. Overall 2 out of 4 bone defects treated with BMP-2-MSCs showed enhancement of bone formation.

### Example 3

### Improved Survival of BMP2 Producing Cells Using Integrating Vectors that Avoid Immune Suppression

Although in Example 2 rapid bone formation was enhanced when the immune response is dampened in Example 3, there can be adverse affects in patients when the immune system is suppressed. Here we rely on the MSC to generate bone without immune response by using alternative and integrating vectors including but not limited plasmid based, Lentiviral and AAV vectors that do not generate as extensive of an immune response. See “Efficient gene delivery into primary cells”; Genecure LLC website http://genecure.com/technology.html.

An important consideration when using viral vectors for clinical gene transfer applications is the presence of pre-existing immunity in the target population. Pre-existing immunity occurs when a patient has been previously exposed to the natural virus, as is common for vectors based on the adenovirus (cause of the common cold) and canarypox virus (a harmless relative of smallpox). Previous exposure the virus prepares the patient's body to quickly mount an immune response should it encounter the virus again. As a result, use of these viral vectors in patients with pre-existing immunity may dampen the effectiveness of the vector due to unwanted immune responses directed at the vector itself. Pre-existing immunity can limit both the ability of the vector to efficiently deliver the gene to target cells as well as limit the duration of gene expression due to immune-mediated destruction of infected cells.

Current clinical gene transfer is hampered by the lack of effective means to deliver genes into primary human cells. The most commonly used gene transfer technologies in clinical studies are retroviral-based vectors derived from murine retroviruses. Unfortunately, these vectors have limited potential for clinical applications due to their inability to infect non-dividing cells. To address this concern, scientists have taken advantage of lentiviruses, which have the natural ability to infect non-dividing mammalian cells. Lentiviral-based vectors including those based on human immunodeficiency virus, HIV-1, have been developed to deliver genes to non-dividing human cells. An alternative is the simian immunodeficiency virus (SIV), a lentivirus family member.

### Safety Profile in Primate Model

In order for viral vectors to be worthy of clinical applications, they must be proven safe in animal models. The primary concern when using retroviruses for human gene transfer applications is the generation of replication-competent retrovirus (RCR). Studies performed in rhesus monkeys to monitor formation of replication-competent virus have validated Lentiviral gene transfer technology as a safe delivery system for, future use in clinical studies.

### Stable Long-Term Expression of Gene

Because lentiviruses permanently integrate into the target cell's genome, lentiviral vectors allow for stable long-term expression of the gene. Numerous reports demonstrate stable expression of reporter genes for greater than nine months. Additionally, unlike commonly used onco-retroviral vectors, where transcriptional silencing of the gene has been observed in numerous reports, no transcriptional silencing has been observed with lentiviral vectors. Thus, use of lentiviral vectors may overcome the challenges hindering current gene transfer technologies.

### Methods

MSC source. The source of MSC can be from various tissues including but not limited to the bone marrow, umbilical cord or a pluripotent stem cell source. These cells can be collected or sourced from commercial entities.

Plasmid-based, lentiviral or AAV vectors and the simian immunodeficiency virus (SIV) were constructed with human cDNA for BMP-2 inserted. MSCs were harvested and plated one day prior to transductions. Standard electroporation techniques are used to incorporate the BMP2 gene. Transduction or transfection were preformed as described previously (Bosch 2006) with minor changes. Upon reaching a density of 36,000 cells/cm² the MSCs were prepared for transduction. To increase cell-viral interactions transductions were performed in reduced medium volumes. Medium was changed with replacement of 32% of normal culture volume of MSC culture medium. Transduction medium was made equaling 20% of normal culture volume with Alpha MEM medium with 2 mM L-glutamine and mixed with 0.72% Genejammer (Agilent Technologies) and allowed to incubate for 5 minutes at room temperature. The virus was then added to the transduction medium and allowed to incubate for 10 minutes at room temperature. After four hours the culture volume was brought up to normal volume with MSC culture medium. MSCs were harvested 24 hours after the transduction. The MSCs were harvested and replated at 36,000 cells/cm² or encapsulated then replated at 36,000 cells/cm². BMP-2 was quantified from harvested medium using a BMP-2 elisa (R&D systems).

### Heterotopic Bone Assay:

Female non-obese diabetic/severely compromised immunodeficient mice (NOD/SCID; 8-12 weeks old; Charles River Laboratories) were injected with 3×10⁶ microencapsulated MSCs either freshly prepared or cryopreserved and thawed from ovine A MSCs and ovine B MSCs. Microbeads were injected into the hind limb quadriceps of 3 mice per group (n=12). Animals were euthanized at 2 weeks and x-rayed. The tissue was then harvested and fixed in formalin.

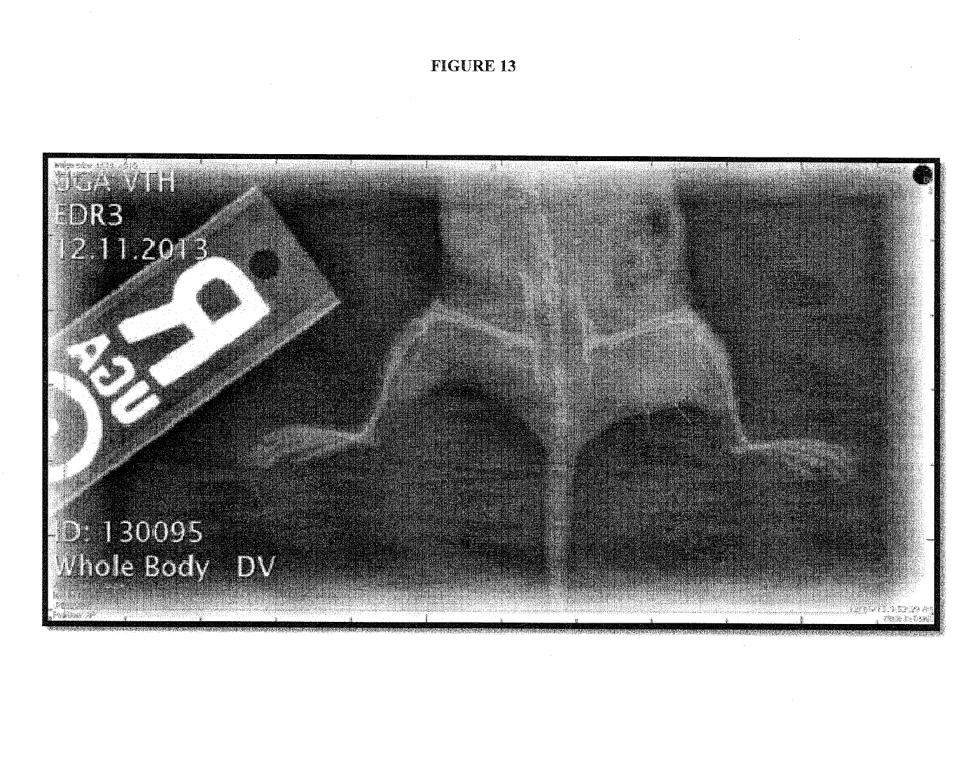

The Lenti system generate extensive BMP2 production in the MSC as assayed by ELISA, HO in rodents (FIG. 13) and is expected in the sheep models described in Example 2.

### Example 4

### Accelerating Bone Formation Using Cell Attracting Microenvironments

Both SDF-1 and BMP-2 are over-expressed to increase endogenous cell migration to the site of injury and reduce inflammatory response in plasmid based or viral based systems. SDF-1 in conjunction with BMP2 will speed the bone repair process by recruiting exogenous cells to the site of injury.

Mesenchymal stem cells (MSC) and hematopoietic stem cells (HSC) can have beneficial effects which alter the inflammatory responses and improve healing. SDF1 and BMP2 expressing cells in microbeads or biological matrix or scaffold such as fibrin will together help to recruit and induce ossification in bone fractures faster than BMP2 alone

Fibrin and other biomaterials that form a scaffold or matrix will be more accepted in clinical applications and should be less inflammatory than PEG based systems.

A composition comprising a population of ossification-inducing microspheres and a population of anti-inflammatory microspheres comprising SDF-1α can be made by a variety of techniques, including those described in Examples 1-3 above.

### Further Examples

In vitro study is undertaken to investigate AAV vs Lenti BMP2 expression levels in rat MSC (Millipore)

Hypothesis: That AAV-BMP2 transduced rat MSC will produce varying levels of BMP2 based upon MOI used.

### Cell Type:

Rat MSC (previously purchased from Millipore)

### Vectors:

Lenti-BMP2 (Cellecta)

AAV-BMP2

### MOI:

Lenti-BMP2-0 and 10 MOI— with 5 ug/mL Polybrene

AAV-BMP2—Want to use 2 different MOI? Without any transduction reagent?

### Experimental Details

### Procedure-Proposed

1. Rat MSC (Millipore) is thawed and expanded.
2. Lentiviral Transduction. On day of transduction cells are harvested and plated at 26,109 cells/cm2.5 ug/mL Polybrene is used. Cells are transduced at 0 and 10 MOI. 1 or 3 wells per timepoint.
3. AAV-BMP2 Transduction. Cells are plated the day before transduction at 26,109 cell/cm2. The following day cells are transduced at 2MOI without any transduction reagent? 1 or 3 wells per timepoint.
4. Media harvests. Media will be collected from the transduced cells at 48, 72, 96, and 120 hours post transduction and frozen at −80.
5. BMP2 ELISA. BMP2 expression levels will be measured via ELISA.

After completion of the above experiment the best AAV-BMP2 MOI is chosen to use with the in vivo immunecompetent rat HO experiment.

### Further Examples

### Exemplary Protocol for Preparing Cross Linked Polyethylene Glycol-DA Microcapsules Containing MSCs

Protocol for photocrosslinking GFP-MSCs IN PEG-DA HYDROGELS USING IRGACURE-2959
1) Place UV lamp in the biosafety hood and turn on for at least 15 minutes before cell encapsulation
2) In a sterile hood, Add 1 ml cell culture media to 100 mg of lyophilized PEG-DA (concentration 10% (w/v) and 10 mg of Irgacure 2959 (concentration 1% (w/v) to make up the crosslinking mixture. Gently mix solution with a pipette to dissolve.
3) Collect and spin down pig GFP-MSCs into a pellet, remove media, add crosslinking mixture to the pellet and gently triturate to resuspend the cell pellet.
4) Dispense 100 μl of the cell suspension to each well of a 24 well glass-bottom plate, and tap gently to facilitate uniform spreading of the cell suspension (alternatively, the wells can be pre-wetted with basal media).
5) Irradiate the cell suspension at 365 nm UV light for 1 minute at room temperature or until gelation occurs.

Different PEG-DA and Irgacure 2959 concentrations can be optimized to maximize cellular viability and modulate hydrogel stiffness.

### MSC Isolation and Culture.

Human bone marrow derived MSC and human umbilical cord derived MSC (Wharton's Jelly) were used. These cells have been quality tested and confirmed via flow cytometry to express multiple markers of MSC by the vendor. Ovine MSCs were isolated previously and have been lineage differentiated to confirm that they are MSC (Mumaw et al. 2011). Briefly, MSCs were isolated from bone marrow aspirates with 0.25 mLs acid citrate dextrose per mL of bone marrow. MSCs were plated by mixing in 3/5 ratio with MSC culture medium:Alpha-Minimum Essential Medium (Gibco), 10% defined fetal bovine serum (Hyclone), 2 mM L-glutamine, 50 U/mL penicillin (Pen), 50 μg/mL streptomycin (Strep; all from Gibco), plating on tissue culture flasks. Cultures were maintained at 37° C. and 5% CO₂.

### Lentiviral Transduction Optimization and BMP2 Quantification.

Custom lentiviruses were constructed with human cDNA for BMP2 under the EF1 alpha promoter and packaged by Cellecta as well as a control construct containing red fluorescent protein (RFP) also under the EF1 alpha promoter. MSCs were harvested with 0.05% trypsin (Gibco) on the day of transduction and resuspended in Alpha-Minimum Essential Medium (Gibco) with 10% defined fetal bovine serum (Hyclone) at 26,109 cells per cm² in 24 well tissue culture treated plates. 5 μg/mL hexadimethrine bromide (Polybrene) (Sigma) was used to increase transduction efficiency and lentivirus was added at multiplicity of infection (MOI) ranging from 0 (no virus, with polybrene) to 50 MOI. After 24 hours, media was replaced with complete MSC culture medium. Media was harvested and frozen at −80° C. from the cells at timepoints ranging from 48 to 120 hours post transduction. RFP lentivirus was used initially to calculate transduction efficiency before using the BMP2-lentivirus. BMP2 was quantified from harvested media using a BMP2 ELISA (R & D Systems).

### Graphical Representation.

Graphs were created in Excel.

### Results

Transduction Efficiency of pR-EF1a-TagRFP-2A-Puro Cellecta Lentivirus in Ovine MSC.

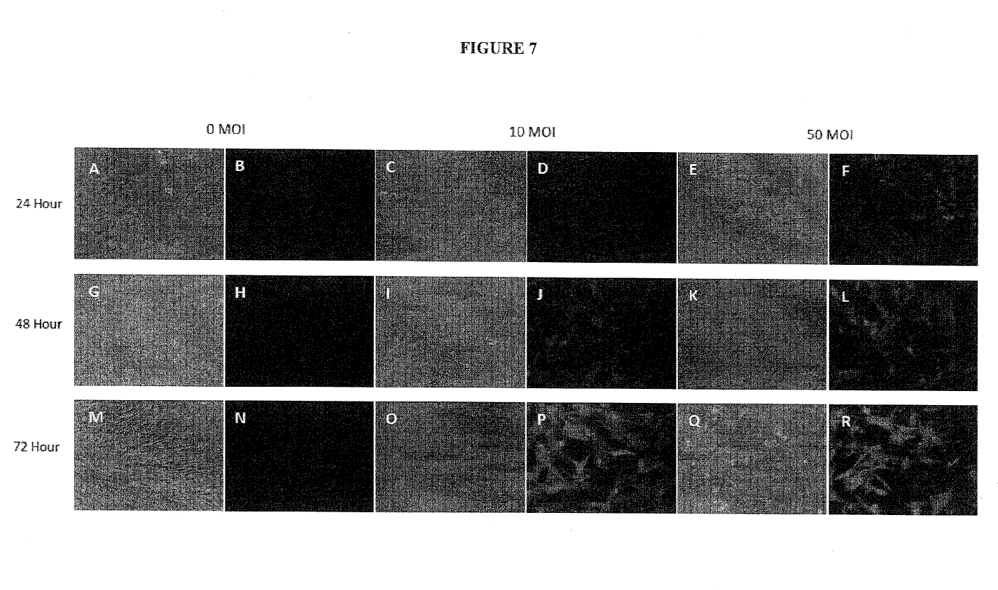

MSCs were transduced at 26,109 cells/cm² at 0 (FIG. 7. A, B, G, H, M, N), 10 (FIG. 7. C, D, I, J, O, P), and 50 MOI (FIG. 1. E, F, K, L, Q, R) with lentivirus and 5 μg/mL hexadimethrine bromide, and phase contrast and fluorescent images were taken at 24 (FIG. 7. A-F), 48 (FIG. 7. G-L), and 72 (FIG. 7. M-R) hours post transduction (100×).

### Conclusion:

Results reported in FIG. 7 indicate that sheep MSCs can be readily transduced with the pR-EF1a-TagRFP-2A-Puro vector with nearly 100% efficiency, especially at higher MOI, and expression of red fluorescent protein occurs as soon as 24 hours post transduction, especially at higher MOI. Based upon these results, 10 MOI was chosen for the next experiments to transduce the cells with the pR-EF1a-BMP2 construct as nearly 100% transduction efficiency was seen after 48 hours (FIG. 7. I, J) with little cell death.

Sheep MSCs Transduced with BMP2.

Sheep bone marrow derived MSCs were transduced with pR-EF1a-BMP2 at multiplicities of infection 0, or 10, as indicated (FIG. 8). Secreted BMP-2 was measured from tissue culture supernatants using a well characterized enzyme-linked immunosorbent assay (ELISA) procedure (R & D Systems), at timepoints 48, 72, and 96 hours following transduction. Secreted BMP2 was assayed in triplicate with median values shown. At 48 hours post-transduction, MSCs produced approximately 19,000 pg/ml BMP2. Levels of BMP2 increased to 103,000 pg/ml at 72 hours, and 153,000 pg/ml at 96 hours following transduction.

### Conclusion:

More BMP2 was detected in sheep supernatants as time progressed, indicating that lentiviral transduction produced equal or in some instances, substantially better results than were previously reported with adenoviral transduction. This is an unexpected result.

### Human Bone Marrow MSCs Secrete Increasing Amounts of BMP2 Post-Transduction.

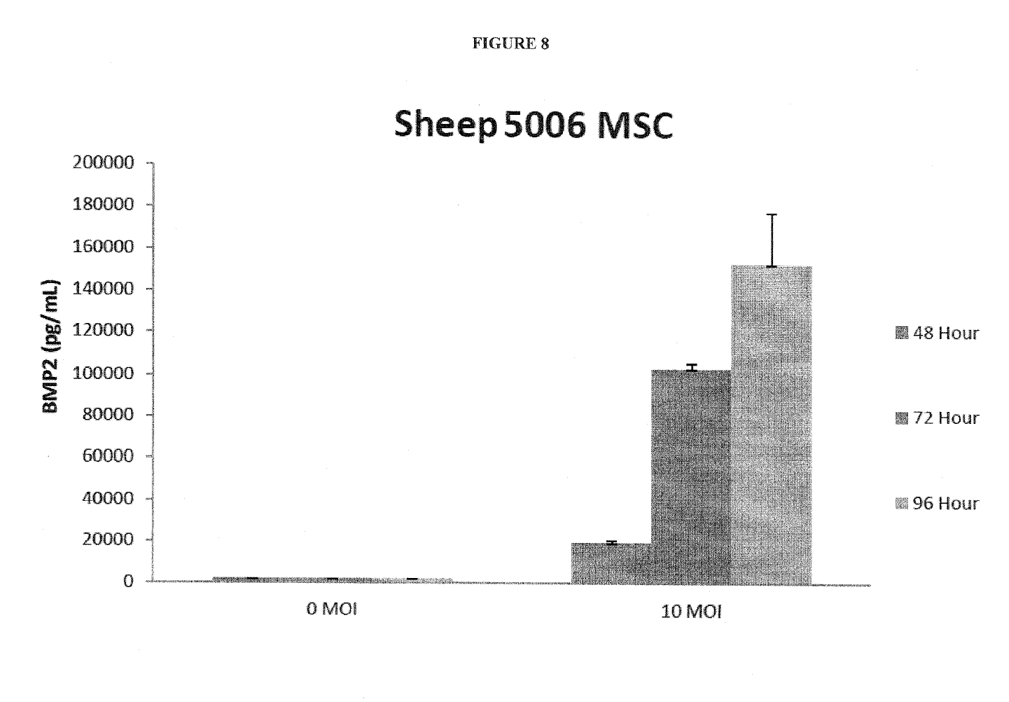

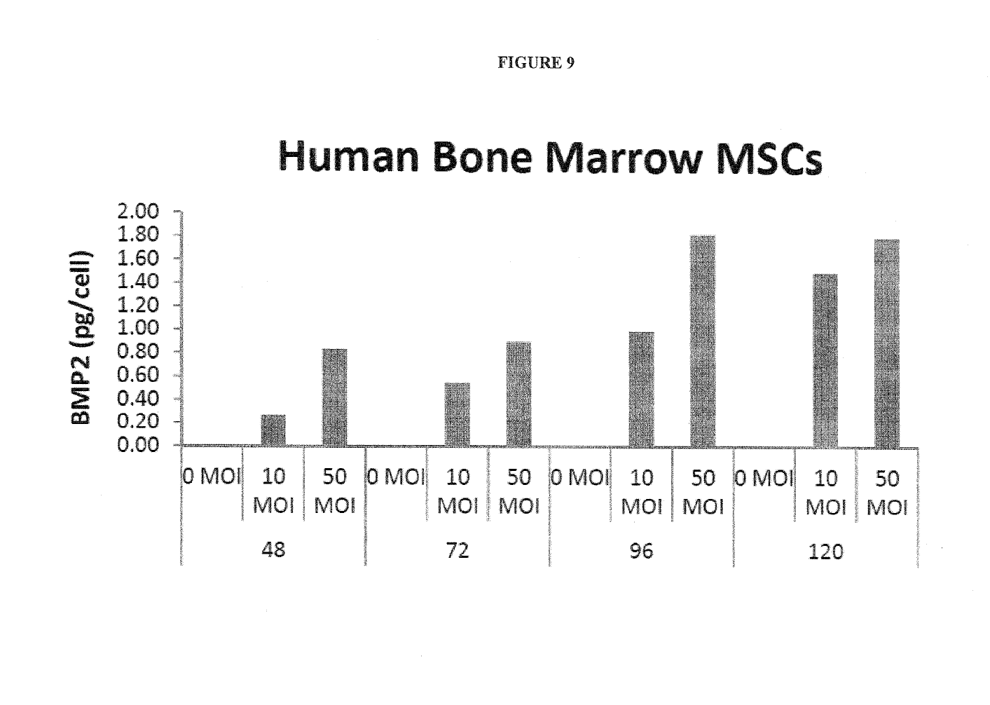

Umbilical (Wharton's Jelly) derived MSCs were transduced with pR-EF1a-BMP2 at multiplicities of infection 0, 10, or 50 as indicated (FIG. 9). Secreted BMP-2 was measured from tissue culture supernatants using a well characterized enzyme-linked immunosorbent assay (ELISA) procedure (R & D Systems), at timepoints 48, 72, 96, and 120 hours following transduction. Amounts of BMP2 increased from approximately 0.2 pg/cell at 10 MOI 24 hours post-transduction, to 1.5-1.8 pg/cell secreted with 10 viral particles per cell or 50 viral particles per cell respectively.

### Conclusion:

As occurred with sheep MSCs, more BMP2 was secreted into the supernatant with time. There was considerably more BMP2 produced with increasing MOI at later timepoints. This further supported the use of 10 MOI in future experiments.

### Human Umbilical MSCs Secrete Greater Amounts of BMP2 Post-Transduction.

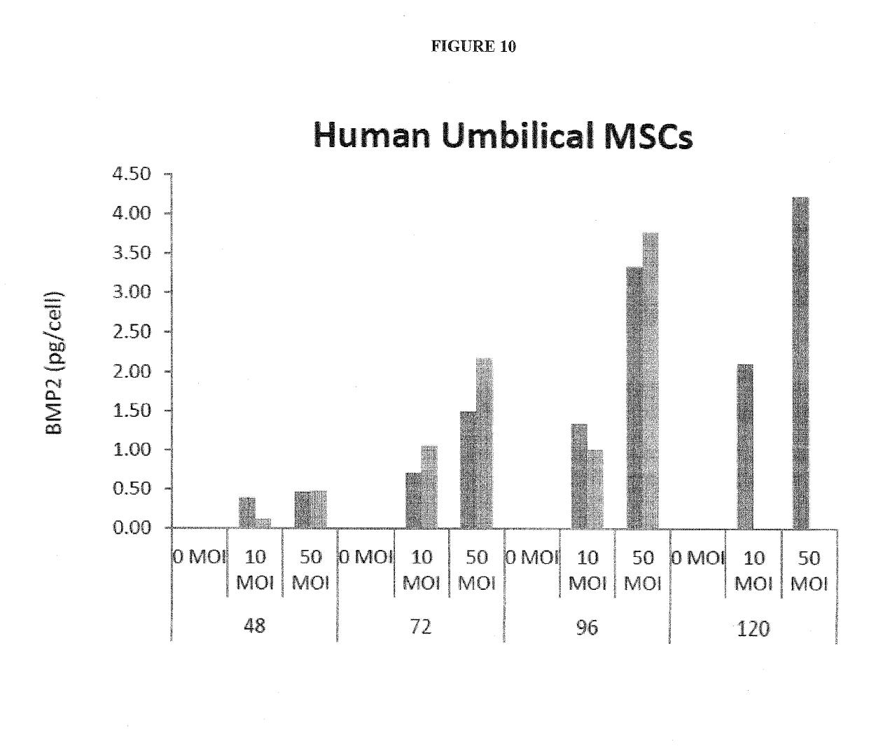

Umbilical (Wharton's Jelly) derived MSCs were transduced with pR-EF1a-BMP2 at multiplicities of infection 0, 10, or 50 as indicated (FIG. 10). Secreted BMP-2 was measured from tissue culture supernatants using a well characterized enzyme-linked immunosorbent assay (ELISA) procedure (R & D Systems), at timepoints 48, 72, 96, and 120 hours following transduction. While initial timepoints produced comparable levels of BMB2 as bone marrow derived MSCs, by 72 hours umbilical cells were producing 1.5 to 2 pg/cell, approximately the same levels produced by bone marrow derived stem cells at 120 hours. The amount of secreted BMP2 continued to increase with time and MOI at levels that were fairly consistent between experiments (red and green bars represent separate transductions). Maximum levels of BMP2 measured at 120 hours post-transduction exceeded 4 pg/cell.

### Conclusion:

Human umbilical MSCs produced the greatest amount of BMP2 per cell of any of the cell lines tested. It is possible that because these cells are derived from an earlier stage of development, a higher global rate of translation results in increased efficiency of BMP2 production.

Comparison of BMP2 Produced from Adeno Vs Lenti Virus.

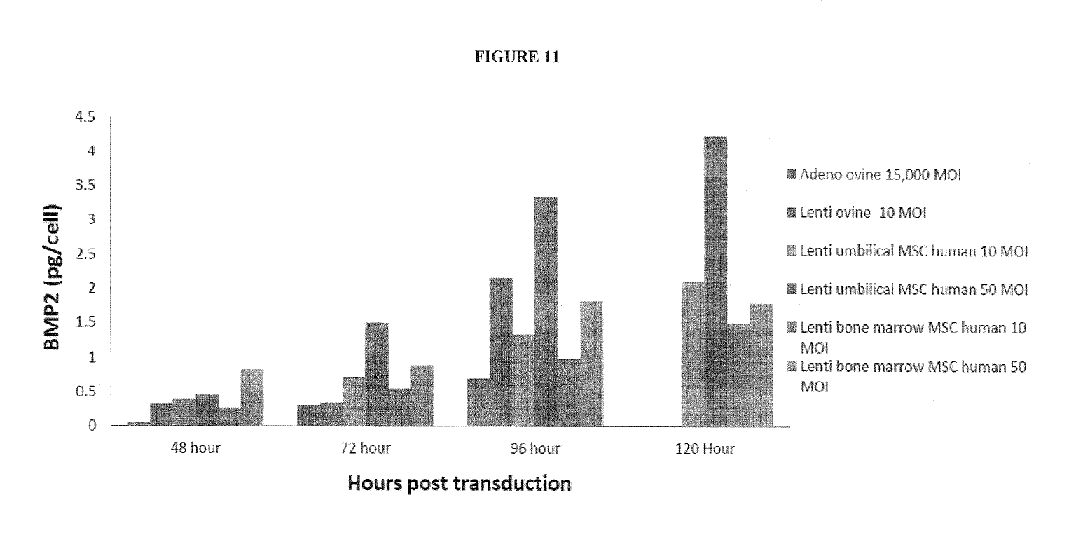

BMP2 production was measured from monolayers using adenoviral and lentiviral constructs in ovine bone marrow MSC, human umbilical MSC, and human bone marrow MSC (FIG. 11). Cells were transduced at 15,000 MOI (adenoviral; ovine MSC), 10 MOI (lentiviral; ovine and human MSC), and 50 MOI (lentiviral; human MSC). Media was harvested from transduced cells at 48, 72, 96, and 120 hours post transduction (except for ovine MSC, no 120 hour sample) and frozen at −80° C. BMP2 was quantified from harvested media using a BMP2 ELISA kit.

### Conclusion:

Based upon this data it was determined that the lentiviral BMP2 is able to produce substantially more BMP2 from transduced cells than the adenoviral BMP2 and that there does not seem to be a tissue specific or species specific response. Also, a much lower

MOI was able to be used in the lentiviral transduced cells than in the adenoviral transduced cells which might lead to less of an immune response during therapy in vivo.

### Example

### Use of Encapsulated Lentiviral BMP2-Expressing Ovine Bone Marrow Derived MSCs (BMP2-MSCs) Repair Long Bone Fractures in Sheep without Exogenous Immunosuppression

Sheep receive injections of encapsulated BMP2-MSCs near a proximal tibial uni-cortical defect. To create this defect sheep are anesthetized and placed in dorsal recumbancy. The proximal aspect of the tibia is clipped and surgically prepared. In a percutaneous approach, the proximal tibial is drilled with a 4.5 mm drill bit aimed at the proximo-dorso-lateral aspect of the bone. The drill is advanced into the bone to penetrate the cortex and the trabecular metaphyseal bone. Three to four mls of encapsulated lentiviral BMP2-expressing ovine bone marrow derived MSCs are delivered into the area of the defect within the drill hole. The skin is closed with skin staples. Tibias are radiographed at 14 and 28 days. Bone formation is observed in the treated tibias of all animals by 28 days.

Conclusion: This experiment demonstrates that the encapsulated lentiviral BMP2-expressing ovine bone marrow derived MSCs, as opposed to the encapsulated adenoviral BMP-2 expressing ovine bone marrow derived MSCs, do not induce a significant immune response, do not require exogenous immunosuppression of the sheep, and therefore allow bone formation.

### Example

### Lentiviral BMP2 Expression Levels are Greater than that of Adenoviral BMP2 Expression Levels

BMP2 production was measured from monolayers using adenoviral and lentiviral constructs in ovine bone marrow MSC, human umbilical MSC, and human bone marrow MSC (FIG. 11). Cells were transduced at 15,000 MOI (adenoviral; ovine MSC), 10 MOI (lentiviral; ovine and human MSC), and 50 MOI (lentiviral; human MSC). Media was harvested from transduced cells at 48, 72, 96, and 120 hours post transduction (except for ovine MSC, no 120 hour sample) and frozen at −80° C. BMP2 was quantified from harvested media using a BMP2 ELISA kit.

Conclusion: Based upon this data, it was determined that the lentiviral BMP2 is able to produce more BMP2 from transduced cells than the adenoviral BMP2 and that there does not seem to be a tissue specific or species specific response. This is not expected when compared to the literature (Blum et al, 2003)(others as listed below) in which adenoviral vectors produced larger amounts of BMP2. Also, a much lower MOI was able to be used in the lentiviral transduced cells than in the adenoviral transduced cells which might lead to less of an immune response in later in vivo experiments.

Potential reasons for unanticipated/unexpected results:
1). Different promoter, EF1a for Lenti and CMV for adeno. Or vector construction. (vector map and sequence attached)
2) Media used had lower serum amount <5% for Lenti studies (unlikely)
3) Cell type better expression with umbilical derived cells than bone marrow derived
4) Because Lenti is an integrating vector it is integrating in favorable sites for expression (this is unlikely).

### Example 7

### Lentiviral BMP2 Expressing MSCs Form Bone in Mammals

### Heterotopic Ossification (HO) In Vivo.

All animal work was completed in accordance with the University of Georgia's IACUC committee. Human MSCs were transduced at an MOI of 10 viral particles per cell with pR-EF1a-BMP2 lentivirus (map of vector, FIG. 12), 24 hours prior to injections into the animals. Eight to twelve week old female immunocompromised NOD/SCID mice (Harlan) were anesthetized with isoflourane (5 animals total), and 3 million virally transduced MSCs were injected via tuberculin syringe into the hindlimb skeletal muscle. A subset of animals was euthanized 7 days post-injection for radiography, with the remainder to be euthanized 14 days post-injection for radiographic and microtomographic analysis of ectopic ossification. Mice formed bone as early as 7 days post injection (FIG. 13, attached) with extensive bone formation by 14 days and mechanical strength similar to contralateral bone by 6 weeks.

In a second model of bone formation, bilateral, critically sized defects (8 mm) are surgically created in femora of 13-week old Nude rats. Limbs are stabilized by custom radiolucent fixation plates that allowed in vivo monitoring with X-ray and microcomputed tomography (microCT). On one femur, varying doses of encapsulated lentiviral BMP2-expressing UMSCs are packed into the defect site and the surgical site closed. The control femur is not treated. Femurs treated with encapsulated lentiviral BMP2-expressing UMSCs form bridging bone in two weeks to four weeks, as compared to no bone formation in the controls. Biomechanical testing in torsion to failure are performed on treated femurs and demonstrate torsional stiffness similar to that of an intact femur within 8 weeks post injury. Mechanical properties exhibit continuously increasing stiffness and torque with increasing dose of cells.

In a third application, sheep with a surgically induced tibial critically sized defect (1 cm to 2 cm) are treated with encapsulated lentiviral BMP2-expressing ovine bone marrow derived MSCs form bridging bone in four to six weeks as compared to controls that form no bone. For biomechanical testing, both ends of the tibia are embedded in 80 ml Paladur and mounted in an Instron 8874 biaxial testing machine. A torsion test is conducted at an agular velocity of 0.5 degrees and a compressive load of 0.05 kN until the fracture point is reached. The contralateral tibia is used as a paired reference. The torsional Moment™ and the torsional stiffness (TS) are calculated from the slope of the torque-angular displacement curves and normalized against the values of the intact contralateral tibia. Biomechanical testing in torsion to failure are performed on treated tibias and demonstrate torsional stiffness similar to that of an intact tibias. Mechanical properties exhibit continuously increasing stiffness and torque with increasing dose of cells.

### REFERENCES FOR THE BACKGROUND OF THE INVENTION

### References

### First Set

Abe, E. (2006). “Function of BMPs and BMP antagonists in adult bone.” Ann N Y Acad Sci 1068: 41-53.
Aggarwal, S. and M. F. Pittenger (2005). “Human mesenchymal stem cells modulate allogeneic immune cell responses.” Blood 105(4): 1815-1822.
Bao, P., A. Kodra, M. Tomic-Canic, M. S. Golinko, H. P. Ehrlich and H. Brem (2009). “The role of vascular endothelial growth factor in wound healing.” J Surg Res 153(2): 347-358.
Bikram, M., C. Fouletier-Dilling, J. A. Hipp, F. Gannon, A. R. Davis, E. A. Olmsted-Davis and J. L. West (2007). “Endochondral bone formation from hydrogel carriers loaded with BMP2-transduced cells.” Ann Biomed Eng 35(5): 796-807.
Bjugstad, K. B., D. E. Redmond, Jr., K. J. Lampe, D. S. Kern, J. R. Sladek, Jr. and M. J. Mahoney (2008). “Biocompatibility of PEG-based hydrogels in primate brain.” Cell Transplant 17(4): 409-415.
Bosch, P., S. L. Pratt and S. L. Stice (2006). “Isolation, characterization, gene modification, and nuclear reprogramming of porcine mesenchymal stem cells.” Biol Reprod 74(1):46-57.
Chang, C. and A. Hemmati-Brivanlou (1998). “Cell fate determination in embryonic ectoderm.” J Neurobiol 36(2): 128-151.
Chen, D., M. Zhao and G. R. Mundy (2004). “Bone morphogenetic proteins.” Growth Factors 22(4): 233-241.
Chin Heng, B., H. Yu and S. Chye Ng (2004). “Strategies for the cryopreservation of microencapsulated cells.” Biotechnol Bioeng 85(2): 202-213.
Dai, L. J., M. R. Moniri, Z. R. Zeng, J. X. Zhou, J. Rayat and G. L. Warnock (2011). “Potential implications of mesenchymal stem cells in cancer therapy.” Cancer Lett 305(1): 8-20.
Dixit, V., R. Darvasi, M. Arthur, K. Lewin and G. Gitnick (1993). “Cryopreserved microencapsulated hepatocytes—transplantation studies in Gunn rats.” Transplantation 55(3): 616-622.
Dragoo, J. L., J. Y. Choi, J. R. Lieberman, J. Huang, P. A. Zuk, J. Zhang, M. H. Hedrick and P. Benhaim (2003). “Bone induction by BMP-2 transduced stem cells derived from human fat.” J Orthop Res 21(4): 622-629.
Guicheux, J., J. Lemonnier, C. Ghayor, A. Suzuki, G. Palmer and J. Caverzasio (2003). “Activation of p38 mitogen-activated protein kinase and c-Jun-NH2-terminal kinase by BMP-2 and their implication in the stimulation of osteoblastic cell differentiation.” J Bone Miner Res 18(11): 2060-2068.
Hare, J. M., J. H. Traverse, T. D. Henry, N. Dib, R. K. Strumpf, S. P. Schulman, G. Gerstenblith, A. N. DeMaria, A. E. Denktas, R. S. Gammon, J. B. Hermiller, Jr., M. A. Reisman, G. L. Schaer and W. Sherman (2009). “A randomized, double-blind, placebo-controlled, dose-escalation study of intravenous adult human mesenchymal stem cells (prochymal) after acute myocardial infarction.” J Am Coll Cardiol 54(24): 2277-2286.
Heng, B. C., Y. J. Yu and S. C. Ng (2004). “Slow-cooling protocols for microcapsule cryopreservation.” J Microencapsul 21(4): 455-467.
Jeon, O., S. J. Song, H. S. Yang, S. H. Bhang, S. W. Kang, M. A. Sung, J. H. Lee and B. S. Kim (2008). “Long-term delivery enhances in vivo osteogenic efficacy of bone morphogenetic protein-2 compared to short-term delivery.” Biochem Biophys Res Commun 369(2): 774-780.
Kawamura, C., M. Kizaki and Y. Ikeda (2002). “Bone morphogenetic protein (BMP)-2 induces apoptosis in human myeloma cells.” Leuk Lymphoma 43(3): 635-639.
Kebriaei, P. and S. Robinson (2011). “Treatment of graft-versus-host-disease with mesenchymal stromal cells.” Cytotherapy 13(3): 262-268.
Kishigami, S. and Y. Mishina (2005). “BMP signaling and early embryonic patterning.” Cytokine Growth Factor Rev 16(3): 265-278.
Koh, J. T., Z. Zhao, Z. Wang, I. S. Lewis, P. H. Krebsbach and R. T. Franceschi (2008). “Combinatorial gene therapy with BMP2/7 enhances cranial bone regeneration.” J Dent Res 87(9): 845-849.
Krebsbach, P. H., K. Gu, R. T. Franceschi and R. B. Rutherford (2000). “Gene therapy-directed osteogenesis: BMP-7-transduced human fibroblasts form bone in vivo.” Hum Gene Ther 11(8): 1201-1210.
Lee, J. Y., D. Musgrave, D. Pelinkovic, K. Fukushima, J. Cummins, A. Usas, P. Robbins, F. H. Fu and J. Huard (2001). “Effect of bone morphogenetic protein-2-expressing muscle-derived cells on healing of critical-sized bone defects in mice.” J Bone Joint Surg Am 83-A(7): 1032-1039.
Li, B. G., T. C. Hua, H. D. Zhang, Y. F. Wang and G. X. Wang (2002). “Cryopreservation and xenotransplantation studies of microencapsulated rat pancreatic islets.” Cryo Letters 23(1): 47-54.
Lin, Z. Q., T. Kondo, Y. Ishida, T. Takayasu and N. Mukaida (2003). “Essential involvement of IL-6 in the skin wound-healing process as evidenced by delayed wound healing in IL-6-deficient mice.” J Leukoc Biol 73(6): 713-721.
Liu, X. Y., J. M. Nothias, A. Scavone, M. Garfinkel and J. M. Millis (2010). “Biocompatibility investigation of polyethylene glycol and alginate-poly-L-lysine for islet encapsulation.” ASAIO J 56(3): 241-245.
Lutolf, M. P., F. E. Weber, H. G. Schmoekel, J. C. Schense, T. Kohler, R. Muller and J. A. Hubbell (2003). “Repair of bone defects using synthetic mimetics of collagenous extracellular matrices.” Nat Biotechnol 21(5): 513-518.
Manieri, N. A. and T. S. Stappenbeck (2011). “Mesenchymal stem cell therapy of intestinal disease: are their effects systemic or localized?” Curr Opin Gastroenterol 27(2): 119-124.
Matthay, M. A., B. T. Thompson, E. J. Read, D. H. McKenna, Jr., K. D. Liu, C. S. Calfee and J. W. Lee (2010). “Therapeutic potential of mesenchymal stem cells for severe acute lung injury.” Chest 138(4): 965-972.
Mayer, F. Q., G. Baldo, T. G. de Carvalho, V. L. Lagranha, R. Giugliani and U. Matte (2010). “Effects of cryopreservation and hypothermic storage on cell viability and enzyme activity in recombinant encapsulated cells overexpressing alpha-L-iduronidase.” Artif Organs 34(5): 434-439.
Mosekilde, L., J. Kragstrup and A. Richards (1987). “Compressive strength, ash weight, and volume of vertebral trabecular bone in experimental fluorosis in pigs.” Calcif Tissue Int 40(6): 318-322.
Mussano, F., G. Ciccone, M. Ceccarelli, I. Baldi and F. Bassi (2007). “Bone morphogenetic proteins and bone defects: a systematic review.” Spine (Phila. Pa. 1976) 32(7): 824-830.
Nauta, A. J., A. B. Kruisselbrink, E. Lurvink, R. Willemze and W. E. Fibbe (2006). “Mesenchymal stem cells inhibit generation and function of both CD34+-derived and monocyte-derived dendritic cells.” J Immunol 177(4): 2080-2087.
Newman, E., A. S. Turner and J. D. Wark (1995). “The potential of sheep for the study of osteopenia: current status and comparison with other animal models.” Bone 16(4 Suppl): 277S-284S.
Nohe, A., E. Keating, P. Knaus and N. O. Petersen (2004). “Signal transduction of bone morphogenetic protein receptors.” Cell Signal 16(3): 291-299.
Olabisi, R. M., Z. Lazard, M. H. Heggeness, K. M. Moran, J. A. Hipp, A. K. Dewan, A. R. Davis, J. L. West and E. A. Olmsted-Davis (2011). “An injectable method for noninvasive spine fusion.” Spine J 11(6): 545-556.
Olabisi, R. M., Z. W. Lazard, C. L. Franco, M. A. Hall, S. K. Kwon, E. M. Sevick-Muraca, J. A. Hipp, A. R. Davis, E. A. Olmsted-Davis and J. L. West (2010). “Hydrogel microsphere encapsulation of a cell-based gene therapy system increases cell survival of injected cells, transgene expression, and bone volume in a model of heterotopic ossification.” Tissue Eng Part A 16(12): 3727-3736.
Osyczka, A. M. and P. S. Leboy (2005). “Bone morphogenetic protein regulation of early osteoblast genes in human marrow stromal cells is mediated by extracellular signal-regulated kinase and phosphatidylinositol 3-kinase signaling.” Endocrinology 146(8): 3428-3437.
Peppas, N. A. and W. H. Yang (1981). “Properties-based optimization of the structure of polymers for contract lens applications.” Contact Intraocul Lens Med J 7(4): 300-314.
Raab, D. M., T. D. Crenshaw, D. B. Kimmel and E. L. Smith (1991). “A histomorphometric study of cortical bone activity during increased weight-bearing exercise.” J Bone Miner Res 6(7): 741-749.
Rabanel, J. M., X. Banquy, H. Zouaoui, M. Mokhtar and P. Hildgen (2009). “Progress technology in microencapsulation methods for cell therapy.” Biotechnol Prog 25(4): 946-963.
Rennekampff, H. O., J. F. Hansbrough, V. Kiessig, C. Dore, M. Sticherling and J. M. Schroder (2000). “Bioactive interleukin-8 is expressed in wounds and enhances wound healing.” J Surg Res 93(1): 41-54.
Sawhney, A. S., C. P. Pathak and J. A. Hubbell (1993). “Interfacial photopolymerization of poly(ethylene glycol)-based hydrogels upon alginate-poly(l-lysine) microcapsules for enhanced biocompatibility.” Biomaterials 14(13): 1008-1016.
Selmani, Z., A. Naji, I. Zidi, B. Favier, E. Gaiffe, L. Obert, C. Borg, P. Saas, P. Tiberghien, N. Rouas-Freiss, E. D. Carosella and F. Deschaseaux (2008). “Human leukocyte antigen-G5 secretion by human mesenchymal stem cells is required to suppress T lymphocyte and natural killer function and to induce CD4+CD25 highFOXP3+ regulatory T cells.” Stem Cells 26(1): 212-222.
Stiegler, P. B., V. Stadlbauer, S. Schaffellner, G. Halwachs, C. Lackner, O. Hauser, F. Iberer and K. Tscheliessnigg (2006). “Cryopreservation of insulin-producing cells microencapsulated in sodium cellulose sulfate.” Transplant Proc 38(9): 3026-3030.
Sun, X., M. Lewandoski, E. N. Meyers, Y. H. Liu, R. E. Maxson, Jr. and G. R. Martin (2000). “Conditional inactivation of Fgf4 reveals complexity of signalling during limb bud development.” Nat Genet 25(1): 83-86.
Thorwarth, M., S. Schultze-Mosgau, P. Kessler, J. Wiltfang and K. A. Schlegel (2005). “Bone regeneration in osseous defects using a resorbable nanoparticular hydroxyapatite.” J Oral Maxillofac Surg 63(11): 1626-1633.
Uccelli, A., F. Benvenuto, A. Laroni and D. Giunti (2011). “Neuroprotective features of mesenchymal stem cells.” Best Pract Res Clin Haematol 24(1): 59-64.
Urist, M. R. (1965). “Bone: formation by autoinduction.” Science 150(698): 893-899.
van der Horst, G., R. L. van Bezooijen, M. M. Deckers, J. Hoogendam, A. Visser, C. W. Lowik and M. Karperien (2002). “Differentiation of murine preosteoblastic KS483 cells depends on autocrine bone morphogenetic protein signaling during all phases of osteoblast formation.” Bone 31(6): 661-669.
Van Lieshout, E. M., G. H. Van Kralingen, Y. El-Massoudi, H. Weinans and P. Patka (2011). “Microstructure and biomechanical characteristics of bone substitutes for trauma and orthopaedic surgery.” BMC Musculoskelet Disord 12: 34.
Wen, X. Z., S. Miyake, Y. Akiyama and Y. Yuasa (2004). “BMP-2 modulates the proliferation and differentiation of normal and cancerous gastric cells.” Biochem Biophys Res Commun 316(1): 100-106.
Williams, A. R., B. Trachtenberg, D. L. Velazquez, I. McNiece, P. Altman, D. Rouy, A. M. Mendizabal, P. M. Pattany, G. A. Lopera, J. Fishman, J. P. Zambrano, A. W. Heldman and J. M. Hare (2011). “Intramyocardial stem cell injection in patients with ischemic cardiomyopathy: functional recovery and reverse remodeling.” Circ Res 108(7): 792-796.
Willie, B. M., R. D. Bloebaum, W. R. Bireley, K. N. Bachus and A. A. Hofmann (2004). “Determining relevance of a weight-bearing ovine model for bone ingrowth assessment.” J Biomed Mater Res A 69(3): 567-576.
Wilson, J. T. and E. L. Chaikof (2008). “Challenges and emerging technologies in the immunoisolation of cells and tissues.” Adv Drug Deliv Rev 60(2): 124-145.
Yamaguchi, A., T. Ishizuya, N. Kintou, Y. Wada, T. Katagiri, J. M. Wozney, V. Rosen and S. Yoshiki (1996). “Effects of BMP-2, BMP-4, and BMP-6 on osteoblastic differentiation of bone marrow-derived stromal cell lines, ST2 and MC3T3-G2/PA6.” Biochem Biophys Res Commun 220(2): 366-371.
Yamamoto, Y. and M. Oelgeschlager (2004). “Regulation of bone morphogenetic proteins in early embryonic development.” Naturwissenschaften 91(11): 519-534.
Yanez, R., A. Oviedo, M. Aldea, J. A. Bueren and M. L. Lamana (2010). “Prostaglandin E2 plays a key role in the immunosuppressive properties of adipose and bone marrow tissue-derived mesenchymal stromal cells.” Exp Cell Res 316(19): 3109-3123.
Zappia, E., S. Casazza, E. Pedemonte, F. Benvenuto, I. Bonanni, E. Gerdoni, D. Giunti, A. Ceravolo, F. Cazzanti, F. Frassoni, G. Mancardi and A. Uccelli (2005). “Mesenchymal stem cells ameliorate experimental autoimmune encephalomyelitis inducing T-cell allergy.” Blood 106(5): 1755-1761.
Zebardast, N., D. Lickorish and J. E. Davies (2010). “Human umbilical cord perivascular cells (HUCPVC): A mesenchymal cell source for dermal wound healing.” Organogenesis 6(4): 197-203.

1. A composition comprising:
(a) one or more biomaterials;
(b) one or more mesenchymal stem cells (MSCs), the MSCs comprising one or more nucleotide sequences encoding one or more bone regeneration proteins,
wherein the one or more nucleotide sequences encoding one or more bone regeneration proteins are operably linked to a promoter; and
(c) an expression vector nucleotide sequence or fragment thereof which expresses said bone regeneration protein(s).
provided that when the composition comprises polyethylene glycol or derivative thereof as a biomaterial the expression vector nucleotide sequence or fragment thereof is not an adenovirus expression vector nucleotide sequence or an adeno-associated virus expression vector nucleotide sequence or fragment thereof.

2. The composition of claim 1, wherein the bone regeneration protein is a bone morphogenetic protein (BMP) and said biomaterial is selected from the group consisting of collagen, fibrin, silk, agarose, alginate, hyaluronan, chitosan, polylactic acid, polyglycolic acid, polylactic-co-glycolic acid, polyethylene glycol, polyethersulfone, a peptide-based biomaterial, a decellularized animal tissue, a ceramic-based biomaterial and mixtures thereof.

3. The composition of claim 1, wherein the bone regeneration protein is BMP-2, BMP-4, BMP-5, BMP-7, or any combination thereof.

4. The composition of claim 1, wherein the bone regeneration protein is a heterodimer of two bone morphogenetic proteins.

5. The composition of claim 4, wherein the heterodimer comprises BMP-2 and BMP-7.

6. The composition of claim 1, further comprising a nucleotide sequence encoding SDF-1α.

7. The composition of claim 1, wherein the composition is a microsphere, gel, putty, or cellular matrix or is a population of microspheres formulated in a gel, putty or cellular matrix.

8. The composition of claim 1, wherein the MSCs are encapsulated by the one or more biomaterials.

9. The composition of claim 1, wherein the MSCs are encapsulated by a polyethylene glycol.

10. The composition of claim 1, wherein the expression vector sequence is a retrovirus, adeno-associated virus, adenovirus, or plasmid sequence.

11. The composition of claim 10, wherein the retrovirus is a lentivirus.

12. The composition of claim 11, wherein the lentivirus is HIV, SIV, or FIV.

13.-16. (canceled)

17. The composition of claim 1 further comprising:
one or more nucleotide sequences encoding SDF-1α, IL-6, IL-8, and/or vascular endothelial growth factor; and/or
a SDF-1α polypeptide, an IL-6 polypeptide, an IL-8 polypeptide, and/or a vascular endothelial growth factor peptide; and/or
prostoglandin E2.

18. A pharmaceutical composition comprising an effective amount of a composition of claim 1 and, optionally, a pharmaceutically acceptable excipient.

19.-21. (canceled)

22. A method of treating a bone or cartilage disorder comprising administering to a subject having the bone or cartilage disorder, a composition of claim 1.

23. The method of claim 22, wherein administering comprises contacting the location of the bone or cartilage defect.

24. (canceled)

25. The method of claim 22, wherein the bone or cartilage disorder is a bone fracture, long bone nonunion, orthopedic soft tissue injury, spinal injury, skeletal and cartilage deficits, bone damage associated with primary bone cancers including osteocarcinoma, congenital bone malformation or nonunion, alveolar bone defects, cranial bone defects, facial bone defects, short bone defects, flat bone defects, irregular bone defects, sesamoid bone defects, cartilage defects, dentoalveolar defects, connective tissue defects, or collagen membrane defects

26.-30. (canceled)

31. A population of ossification-inducing microspheres, the microspheres comprising:
(a) one or more biomaterials; and
(b) one or more mesenchymal stem cells (MSCs) that are encapsulated by and that have been propagated on a scaffolding comprising one or more of biomaterials, wherein the cryopreserved mesenchymal stem cells (MSCs):
(1) have been transfected with an adenovirus-based vector comprising a nucleotide sequence which encodes a bone regeneration protein; or
(2) have been transduced with a retrovirus-based vector comprising a nucleotide sequence which encodes a bone regeneration protein; or
(3) have been transfected with a plasmid comprising a cDNA which encodes a bone regeneration protein; or
(4) have undergone transposon mutagenesis which introduced into their chromosomes a nucleotide sequence which encodes a bone regeneration protein.

32.-43. (canceled)

44. A composition comprising a population of ossification-inducing microspheres and a population of anti-inflammatory microspheres,
wherein each of the ossification-inducing microspheres comprises:
(a) one or more biomaterials selected from the group consisting of collagen, fibrin, silk, agarose, alginate, hyaluronan, chitosan, polylactic-co-glycolic acid, polyethylene glycol, polyethersulfone, a peptide-based biomaterial, a ceramic-based biomaterial and mixtures thereof; and
(b) one or more mesenchymal stem cells (MSCs) that are encapsulated by and that have been propagated on a scaffolding comprising one or more of the biomaterials, wherein the mesenchymal stem cells (MSCs):
(1) have been transfected with an adenovirus-based vector comprising a nucleotide sequence which encodes a bone regeneration protein; or
(2) have been transfected with a retrovirus-based vector comprising a nucleotide sequence which encodes a bone regeneration protein; or
(3) have been transfected with a plasmid comprising a cDNA which encodes a bone regeneration protein; or
(4) have undergone transposon mutagenesis which introduced into their chromosomes a nucleotide sequence which encodes a bone regeneration protein; and
wherein each of the anti-inflammatory microspheres comprises:
(a) one or more biomaterials selected from the group consisting of collagen, fibrin, silk, agarose, alginate, hyaluronan, chitosan, polylactic-co-glycolic acid, polyethylene glycol, polyethersulfone, a peptide-based biomaterial, a ceramic-based biomaterial and mixtures thereof; and
(b) one or more mesenchymal stem cells (MSCs) that are encapsulated by and that have been propagated on a scaffolding comprising one or more of the biomaterials, wherein the cryopreserved mesenchymal stem cells (MSCs):
(1) have been transfected with an adenovirus-based vector comprising a nucleotide sequence which encodes SDF-1α; or
(2) have been transfected with a retrovirus-based vector comprising a nucleotide sequence which encodes SDF-1α; or
(c) have been transfected with a plasmid comprising a cDNA which encodes SDF-1α; or
(d) have undergone transposon mutagenesis which introduced into their chromosomes a nucleotide sequence which encodes SDF-1α;
wherein the types of biomaterials and mesenchymal stem cells which comprise the ossification-inducing microspheres and the anti-inflammatory microspheres may be the same or different.

45.-70. (canceled)

71. A composition comprising:
(a) one or more biomaterials selected from the group consisting of glycosaminoglycan, silk, fibrin, a gelatinous support protein matrix, a peptide hydrogel, decelluarized animal tissue, poly-ethyleneglycol (PEG), polyethylene glycol diacrylate (PEG-DA), polyhydroxy ethyl methacrylate, polyvinyl alcohol, polyacrylamide, poly(N-vinyl pyrolidone) (polyvinylpyrrolidone), poly lactic acid (PLA), poly glycolic acid (PGA), poly lactic-co-glycolic acid (PLGA), poly e-carpolactone (PCL), polyethylene oxide, polyethylene oxide dicaprylate, poly propylene fumarate (PPF), poly acrylic acid (PAA), hydrolysed polyacrylonitrile, polyacrylamide, polyacrylic acid, polymethacrylic acid, polyethylene amine, alginic acid, pectinic acid, carboxy methyl cellulose, hyaluronic acid, heparin, heparin sulfate, chitosan, carboxymethyl chitosan, chitin, pullulan, xyloglucan, gellan, carbopol, pluronics, triblock polymer systems, xanthan, collagen, gelatin, carboxymethyl starch, carboxymethyl dextran, chondroitin sulfate, cationic guar, and cationic starch, or a salt or ester thereof, tissue obtained from a patient or subject to be treated or a mixture thereof
(b) one or more mesenchymal stem cells (MSCs), the MSCs comprising one or more nucleotide sequences encoding one or more bone regeneration proteins,
wherein the one or more nucleotide sequences encoding one or more bone regeneration proteins are operably linked to a promoter; and
(c) an expression vector nucleotide sequence or fragment thereof which expresses said bone regeneration protein(s).

72.-87. (canceled)


SEQUENCE LISTING (7 sequences)

SEQ ID NO: 1 - DNA, 3150 bp, Homo sapiens
ccacaaagggcacttggccccagggctaggagagcgaggggagagcacagccacccgcctcggcggcccgggactcggctcgactcgccggagaatgcgcccgaggacgacggggcgccagagccgcggtgctttcaactggcgagcgcgaatgggggtgcactggagtaaggcagagtgatgcgggggggcaactcgcctggcaccgagatcgccgccgtgcccttccctggacccggcgtcgcccaggatggctgccccgagccatgggccgcggcggagctagcgcggagcgcccgaccctcgacccccgagtcccggagccggccccgcgcggggccacgcgtccctcgggcgctggttcctaaggaggacgacagcaccagcttctcctttctcccttcccttccctgccccgcactcctccccctgctcgctgttgttgtgtgtcagcacttggctggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaacaaacaaacaaaaaaaccccaccccagttgacactttaatatttcccaatgaagactttatttatggaatggaatggaaaaaaaaacagctattttgaaaatatatttatatctacgaaaagaagttgggaaaacaaatattttaatcagagaattattccttaaagatttaaaatgtatttagttgtacattttatatgggttcaaccccagcacatgaagtataatggtcagatttattttgtatttatttactattataaccactttttaggaaaaaaatagctaatttgtatttatatgtaatcaaaagaagtatcgggtttgtacataattttccaaaaattgtagttgttttcagttgtgtgtatttaagatgaaaagtctacatggaaggttactctggcaaagtgcttagcacgtttgcttttttgcagtgctactgttgagttcacaagttcaagtccagaaaaaaaaagtggataatccactctgctgactttcaagattattatattattcaattctcaggaatgttgcagagtgattgtccaatccatgagaatttacatccttattaggtggaatatttggataagaaccagacattgctgatctattatagaaactctcctcctgccccttaatttacagaaagaataaagcaggatccatagaaataattaggaaaacgatgaacctgcaggaaagtgaatgatggtttgttgttcttctttcctaaattagtgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggcatttcattctataaagtc

SEQ ID NO: 2 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 3 - DNA, 4049 bp, Homo sapiens
agcgcgtaccactctggcgctcccgaggcggcctcttgtgcgatccagggcgcacaaggctgggagagcgccccggggcccctgctatccgcgccggaggttggaagagggtgggttgccgccgcccgagggcgagagcgccagaggagcgggaagaaggagcgctcgcccgcccgcctgcctcctcgctgcctccccggcgttggctctctggactcctaggcttgctggctgctcctcccacccgcgcccgcctcctcactcgccttttcgttcgccggggctgctttccaagccctgcggtgcgcccgggcgagtgcggggcgaggggcccggggccagcaccgagcagggggcgggggtccgggcagagcgcggccggccggggaggggccatgtctggcgcgggcgcagcggggcccgtctgcagcaagtgaccgagcggcgcggacggccgcctgccccctctgccacctggggcggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatgaaaatggttaggacgttacagatatattttcctaaacaatttatccccatttctcggtttatcctgatgcgtaaacagaagctgtgtcaagtggagggcggggaggtccctctccattccctacagttttcatcctgaggcttgcagaggcccagtgtttaccgaggtttgcccaaatccaagatctagtgggaggggaaagagcaaatgtctgctccgaggagggcggtgtgttgatctttggaggaaaaatatgttctgttgttcagctggatttgccgtggcagaaatgaaactaggtgtgtgaaatacccgcagacatttgggattggcttttcacctcgccccagtggtagtaaatccatgtgaaattgcagaggggacaaggacagcaagtaggatggaacttgcaactcaaccctgttgttaagaagcaccaatgggccgggcacagtagctcccacctgtaatcccagcactttgggaggctgaggtgggcggatcatttgaggtcaggagttcgagaccagcctggccaacatggtgaaaccccatctctactaaaaatacaaaaattagccgggcatggtggcacgcacctgtaatcccagctactctggaggctgaggcaggagaattgcttgaaccccagaggtggaggttgcagtgagccaagatcgtcccactgcactccagcttgggtgacaaaacaagactccatctcaaaagaaaaaaaaaacagcaccaatgaagcctagttctccacgggagtggggtgagcaggagcactgcacatcgccccagtggaccctctggtctttgtctgcagtggcattccaaggctgggccctggcaagggcacccgtggctgtctcttcatttgcagaccctgatcagaagtctctgcaaacaaatttgctccttgaattaagggggagatggcataataggaggtctgatgggtgcaggatgtgctggacttacattgcaaatagaagccttgttgagggtgacatcctaaccaagtgtcccgatttggaggtggcatttctgacgtggctcttggtgtaagcctgccttgccttggctggtgagtcccataaatagtatgcactcagcctccggccacaaacacaaggcctaggggagggctagactgtctgcaaacgttttctgcatctgtaaagaaaacaaggtgatcgaaaactgtggccatgtggaacccggtcttgtgggggactgtttctccatcttgactcagacagttcctggaaacaccggggctctgtttttattttctttgatgtttttcttctttagtagcttgggctgcagcctccactctctagtcactggggaggagtattttttgttatgtttggtttcatttgctggcagagctggggctttttgtgtgatccctcttggtgtgagttttctgacccaaccagcctctggttagcatcatttgtacatttaaacctgtaaatagttgttacaaagcaaagagattatttatttccatccaaagctcttttgaacacccccccccctttaatccctcgttcaggacgatgagcttgctttccttcaacctgtttgttttcttatttaagactatttattaatggttggaccaatgtactcacagctgttgcgtcgagcagtccttagtgaaaattctgtataaatagacaaaatgaaaagggtttgaccttgcaataaaaggagacgtttggttctggcaaaaaaaaaaaaaaaaaa

SEQ ID NO: 4 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 5 - DNA, 1188 bp, Canis familiaris
atggtggccgggacccgctgtcttctagcgttgctgcttccccaggtcctcctgggcggcgcggccggtctcattacggagctgggccggaggaagttcgcggcatctcctggccgctccccgtcccagccctcggacgaagtcctgagcgagttcgagttgcggctgctcagcatgttcggcctgaagcggagacccacccccagcagggacgccgtggtgcccccctacatgctggacctgtaccgccggcactcgggccagccgggcgcaccggccccggaccaccggctggagagggcagccagcctggccaacaccgtgcgcagcttccaccacgaagaatctttggaagaactgccagaaatgagtgggaaaacaacccggagattcttctttaatttaacttctatccccactgacgagtttatcacctcagcagaacttcaggtttttcgggaacagatgcaggaacctttggaaaacgatagcaatttccatcaccgaattaatatttatgaaattataaaacctgcagcagccaacttgaagttccccgtgaccagacttttggacaccaggttggtgaatcagaacgcaagcaggtgggagagctttgatgtcacccctgctgtgatgaggtggacggcacaggggcttgccaaccacggatttgtggtggaagtgacccacttggaggagaatcaaggtgtctccaagagacacgtcaggattagcaggtctttgcaccaagacgagcacagctggtcacaaatcaggccactgctagtaacgtttggccacgatgggaaaggacaccccctccacaaaagagaaaagcgtcaagcaaaacacaaacagcgcaaacgccttaagtccagctgtaagagacaccctttgtacgtggacttcagtgacgtggggtggaatgactggatcgtagctcccccggggtaccacgccttttattgccatggggaatgcccctttcccctggcagatcacctgaactccactaaccacgccattgttcagacgttggtcaactcggttaattctaagatccccaaggcgtgctgtgtaccaacagaactcagtgctatctccatgctgtaccttgatgaaaacgaaaaggttgtattaaagaactatcaggacatggttgtggagggttgtgggtgtcgttag

SEQ ID NO: 6 - protein, 395 aa, Canis familiaris
MVAGTRCLLALLLPQVLLGGAAGLITELGRRKFAASPGRSPSQPSDEVLSEFELRLLSMFGLKRRPTPSRDAVVPPYMLDLYRRHSGQPGAPAPDHRLERAASLANTVRSFHHEESLEELPEMSGKTTRRFFFNLTSIPTDEFITSAELQVFREQMQEPLENDSNFHHRINIYEIIKPAAANLKFPVTRLLDTRLVNQNASRWESFDVTPAVMRWTAQGLANHGFVVEVTHLEENQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 7 - DNA, 7836 bp, Artificial Sequence
  Lentiviral Vector pR-EF1-BMP2
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttttaaaagaaaaggggggattggggggtacagtgcaggggaaagaatagtagacataatagcaacagacatacaaactaaagaattacaaaaacaaattacaaaaattcaaaattttatcgatggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagccaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggacttggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc